(19) United States
(12) Patent Application Publication
(10) Pub. No.: US 2022/0160419 A1
(43) Pub. Date: May 26, 2022

(54) ELECTROSURGICAL DEVICES AND SYSTEMS HAVING ONE OR MORE POROUS ELECTRODES

(71) Applicant: Apyx Medical Corporation, Clearwater, FL (US)

(72) Inventors: Eric J. Hoegstrom, Dunedin, FL (US); Shawn D. Roman, Safety Harbor, FL (US)

(21) Appl. No.: 17/425,755

(22) PCT Filed: Jan. 27, 2020
(86) PCT No.: PCT/US20/15201

§ 371 Date: Jul. 26, 2021

Related U.S. Application Data
(60) Provisional application No. 62/797,846, filed on Jan. 28, 2019.

(51) Int. Cl. A61B 18/14; A61B 18/16

(52) U.S. Cl. CPC A61B 18/1442; A61B 2018/00065; A61B 18/16

(57) ABSTRACT

An electrosurgical apparatus is provided having a shaft, a handle, and at least one porous electrode. The shaft is coupled to the handle and the at least one porous electrode is coupled to a distal tip of the shaft. The at least one porous electrode conducts energy provided to the distal tip and enables fluid provided to the distal tip to pass or flow through the porous structure of the at least one electrode, such that the electrosurgical energy and the fluid are simultaneously applied to patient tissue adjacent to the at least one porous electrode. The shaft is rotatable relative to the handle of the electrosurgical apparatus to change the orientation of the at least one porous electrode relative to the handle. The shaft is extendable or retractable relative to the handle to increase or decrease the distance between the at least one porous electrode and the handle

### PRIORITY

This application claims priority to U.S. Provisional Patent Application Ser. No. 62/797,846, filed Jan. 28, 2019, entitled “ELECTROSURGICAL DEVICES AND SYSTEMS HAVING ONE OR MORE POROUS ELECTRODES”, the contents of which are hereby incorporated by reference in its entirety.

### BACKGROUND

### Field

The present disclosure relates generally to electrosurgery and electrosurgical systems and apparatuses, and more particularly, to electrosurgical devices and systems having one or more porous electrodes.

### Description of the Related Art

Today, electrosurgery is one of the widely used surgical modalities for treating tissue abnormalities. Electrosurgical devices fall into one of two categories: monopolar devices and bipolar devices. Generally, surgeons are trained in the use of both monopolar and bipolar electrosurgical techniques, and essentially all operating rooms will be found equipped with the somewhat ubiquitous instrumentality for performing electrosurgery.

Monopolar electrosurgical devices typically comprise an electrosurgical probe having a first or “active” electrode extending from one end. The electrosurgical probe is electrically coupled to an electrosurgical generator, which provides a high frequency electric current. A remote control switch is attached to the generator and commonly extends to a foot switch located in proximity to the operating theater. During an operation, a second or “return” electrode, having a much larger surface area than the active electrode, is positioned in contact with the skin of the patient. The surgeon may then bring the active electrode in close proximity to the tissue and activate the foot control switch, which causes electrical current to arc from the distal portion of the active electrode and flow through tissue to the larger return electrode.

For the bipolar modality, no return electrode is used. Instead, a second electrode is closely positioned adjacent to the first electrode, with both electrodes being attached to an electrosurgical probe. As with monopolar devices, the electrosurgical probe is electrically coupled to an electrosurgical generator. When the generator is activated, electrical current arcs from the end of the first electrode to the end of the second electrode, flowing through the intervening tissue. In practice, several electrodes may be employed, and depending on the relative size or locality of the electrodes, one or more electrodes may be active.

Whether arranged in a monopolar or bipolar fashion, the active electrode may be operated to either cut tissue or coagulate tissue. When used to cut tissue, the electrical arcing and corresponding current flow results in a highly intense, but localized heating, sufficient enough to break intercellular bonds, resulting in tissue severance. When used to coagulate tissue, the electrical arcing results in a low level current that denatures cells to a sufficient depth without breaking intercellular bonds, i.e., without cutting the tissue.

Whether tissue is cut or coagulated mainly depends on the geometry of the active electrode and the nature of the electrical energy delivered to the electrode. In general, the smaller the surface area of the electrode in proximity to the tissue, the greater the current density (i.e., the amount of current distributed over an area) of the electrical arc generated by the electrode, and thus the more intense the thermal effect, thereby cutting the tissue. In contrast, the greater the surface area of the electrode in proximity to the tissue, the less the current density of the electrical arc generated by the electrode, thereby coagulating the tissue. Thus, if an electrode having both a broad side and a narrow side is used, e.g., a spatula, the narrow side of the electrode can be placed in proximity to the tissue in order to cut it, whereas the broad side of the electrode can be placed in proximity to the tissue in order to coagulate it. With respect to the characteristics of the electrical energy, as the crest factor (peak voltage divided by root mean squared (RMS)) of the electrical energy increases, the resulting electrical arc generated by the electrode tends to have a tissue coagulation effect. In contrast, as the crest factor of the electrical energy decreases, the resulting electrical arc generated by the electrode tends to have a cutting effect. The crest factor of the electrical energy is typically controlled by controlling the duty cycle of the electrical energy. For example, to accentuate tissue cutting, the electrical energy may be continuously applied to increase its RMS average to decrease the crest factor. In contrast, to accentuate tissue coagulation, the electrical energy may be pulsed (e.g., at a 10 percent duty cycle) to decrease its RMS average to increase the crest factor.

Notably, some electrosurgical generators are capable of being selectively operated in so-called “cutting modes” and “coagulation modes.” This, however, does not mean that the active electrode that is connected to such electrosurgical generators will necessarily have a tissue cutting effect if operated in the cutting mode or similarly will have a tissue coagulation effect if operated in the coagulation mode, since the geometry of the electrode is the most significant factor in dictating whether the tissue is cut or coagulated. Thus, if the narrow part of an electrode is placed in proximity to tissue and electrical energy is delivered to the electrode while in a coagulation mode, the tissue may still be cut.

There are many medical procedures in which tissue is cut or carved away for diagnostic or therapeutic reasons. For example, during hepatic transection, one or more lobes of a liver containing abnormal tissue, such as malignant tissue or fibrous tissue caused by cirrhosis, are cut away. There exist various modalities, including mechanical, ultrasonic, and electrical (which includes RF energy), that can be used to effect resection of tissue. Whichever modality is used, extensive bleeding can occur, which can obstruct the surgeon's view and lead to dangerous blood loss levels, requiring transfusion of blood, which increases the complexity, time, and expense of the resection procedure. To prevent extensive bleeding, hemostatic mechanisms, such as blood inflow occlusion, coagulants, and energy coagulation (e.g., electrosurgical coagulation or argon-beam coagulation), can be used.

In the case where an electrosurgical coagulation means is used, the bleeding can be treated or avoided by coagulating the tissue in the treatment areas with an electro-coagulator that applies a low level current to denature cells to a sufficient depth without breaking intercellular bonds, i.e., without cutting the tissue. Because of their natural coagulation capability, ease of use, and ubiquity, electrosurgical modalities are often used to resect tissue.

During a typical electrosurgical resection procedure, electrical energy can be conveyed from an electrode along a resection line in the tissue. The electrode may be operated in a manner that incises the tissue along the resection line, or coagulates the tissue along the resection line, which can then be subsequently dissected using the same coagulation electrode or a separate tissue dissector to gradually separate the tissue. In the case where an organ is resected, application of RF energy divides the parenchyma, thereby skeletalizing the organ, i.e., leaving vascular tissue that is typically more difficult to cut or dissect relative to the parenchyma.

When a blood vessel is encountered, RF energy can be applied to shrink the collagen in the blood vessel, thereby closing the blood lumen and achieving hemostasis. The blood vessel can then be mechanically transected using a scalpel or scissors without fear of blood loss. In general, for smaller blood vessels less than 3 mm in diameter, hemostasis may be achieved within 10 seconds, whereas for larger blood vessels up to 5 mm in diameter, the time required for hemostasis increases to 15-20 seconds. During or after resection of the tissue, RF energy can be applied to any “bleeders” (i.e., vessels from which blood flows or oozes) to provide complete hemostasis for the resected organ.

When electrosurgically resecting tissue, care must be taken to prevent the heat generated by the electrode from charring the tissue, which generates an undesirable odor, results in tissue becoming stuck on the electrosurgical probe, and most importantly, increases tissue resistance, thereby reducing the efficiency of the procedure. Adding an electrically conductive fluid, such as saline, to the electrosurgery site cools the electrode and keeps the tissue temperature below the water boiling point (100° C.), thereby avoiding smoke and reducing the amount of charring.

Although the application of electrically conductive fluid to the electrosurgery site generally increases the efficiency of the RF energy application, energy applied to an electrode may rapidly diffuse into fluid that has accumulated and into tissue that has already been removed. As a result, if the fluid and removed tissue is not effectively aspirated from the tissue site, the electrosurgery may either be inadequately carried out, or a greater than necessary amount of energy must be applied to the electrode to perform the surgery. Increasing the energy used during electrosurgery increases the chance that adjacent healthy tissues may be damaged. At the same time that fluid accumulation is avoided, care must be taken to ensure that fluid is continuously flowed to the tissue site to ensure that tissue charring does not take place. For example, if flow of the fluid is momentarily stopped, e.g., if the port on the fluid delivery device becomes clogged or otherwise occluded, RF energy may continue to be conveyed from the electrode, thereby resulting in a condition where tissue charring may occur.

There, thus, remains a need to provide a more efficient means for electrosurgically resecting vascularized tissue, while preventing tissue charring and maintaining hemostasis at the treatment site.

### SUMMARY

The present disclosure relates to devices and systems having one or more porous electrodes.

In one aspect of the present disclosure, an electrosurgical apparatus is provided having a handle, a shaft, and at least one electrode. The shaft is coupled to the handle and the at least one porous electrode is disposed on a distal tip of the shaft. The porous electrode is configured to conduct electrosurgical energy provided to the distal tip to be applied to patient tissue disposed adjacent to the electrode. Furthermore, the porous electrode is configured to enable a fluid provided to the distal tip, such as saline, to pass through the porous structure of the electrode and to be provided to the patient tissue adjacent to the electrode.

In another aspect of the present disclosure, the electrosurgical apparatus is configured as a monopolar device having a single electrode.

In another aspect of the present disclosure, the electrosurgical apparatus is configured as a bipolar device having a first electrode and a second electrode.

In another aspect of the present disclosure, the shaft of the electrosurgical apparatus is rotatable relative to a handle of the electrosurgical apparatus to change the orientation of the electrode with respect to the handle.

In another aspect of the present disclosure, the shaft of the electrosurgical apparatus is extendable or retractable relative to the handle of the electrosurgical apparatus to increase or decrease the distance between the electrode and the handle.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is an illustration of an exemplary electrosurgical system including an electrosurgical apparatus in accordance with an embodiment of the present disclosure;

FIG. 1B is an exploded perspective view of the electrosurgical apparatus of the system of FIG. 1A in accordance with an embodiment of the present disclosure;

FIG. 1C is a side view of the electrosurgical apparatus of FIG. 1A with a portion of the housing removed in accordance with an embodiment of the present disclosure;

FIG. 1D is a partial cross-section view of the electrosurgical apparatus of FIG. 1A in accordance with an embodiment of the present disclosure;

FIGS. 1E, 1F, 1G illustrate the operation of a flow control slider of the electrosurgical apparatus of FIG. 1A in accordance with an embodiment of the present disclosure;

FIGS. 1H and 1I illustrate a shaft of the electrosurgical apparatus of FIG. 1A in accordance with an embodiment of the present disclosure;

FIGS. 1J and 1K illustrate an electrode of the electrosurgical apparatus of FIG. 1A in accordance with an embodiment of the present disclosure;

FIG. 1L illustrate the electrode of FIGS. 1J and 1K coupled to a molded cap of the electrosurgical apparatus of FIG. 1A in accordance with an embodiment of the present disclosure;

FIGS. 1M and 1N illustrate the distal end of the electrosurgical apparatus of FIG. 1A in accordance with an embodiment of the present disclosure;

FIGS. 1O and 1P are cross-section views of the distal end of the electrosurgical apparatus of FIG. 1A in accordance with an embodiment of the present disclosure;

FIGS. 1Q, 1R, and 1S are side cross-section views of the electrosurgical apparatus of FIG. 1A in accordance with an embodiment of the present disclosure;

FIG. 1T illustrates an electrode having a concave surface for use with the electrosurgical apparatus of FIG. 1A in accordance with an embodiment of the present disclosure;

FIGS. 1U, 1V, and 1W illustrate an electrode having convex surfaces for use with the electrosurgical apparatus of FIG. 1A in accordance with an embodiment of the present disclosure;

FIG. 2A is an illustration of an exemplary electrosurgical system including another electrosurgical apparatus in accordance with an embodiment of the present disclosure;

FIG. 2B is a perspective view of the electrosurgical apparatus of the system of FIG. 2A in accordance with an embodiment of the present disclosure;

FIG. 2C is an exploded perspective view of the electrosurgical apparatus of FIG. 2A in accordance with an embodiment of the present disclosure;

FIG. 2D is a side view of the electrosurgical apparatus of FIG. 1A with a portion of the housing removed in accordance with an embodiment of the present disclosure;

FIGS. 2E and 2F illustrate a connector component of the electrosurgical apparatus of FIG. 2A in accordance with an embodiment of the present disclosure;

FIG. 2G is a partial cross-section view of the electrosurgical apparatus of FIG. 2A in accordance with an embodiment of the present disclosure;

FIG. 2H is a partial view of the electrosurgical apparatus of FIG. 2A with a portion of the housing removed in accordance with an embodiment of the present disclosure;

FIG. 2I is a partial cross-section view of the electrosurgical apparatus of FIG. 2A in accordance with an embodiment of the present disclosure;

FIGS. 2J and 2K illustrate the connector components of FIGS. 2E and 2F and the shaft of the electrosurgical apparatus of FIG. 2A in accordance with an embodiment of the present disclosure;

FIG. 2L is a perspective view of a tube of the shaft of the electrosurgical apparatus of FIG. 2A in accordance with an embodiment of the present disclosure;

FIGS. 2M and 2N illustrate the electrodes of the electrosurgical apparatus of FIG. 2A in accordance with an embodiment of the present disclosure;

FIGS. 2O, 2P, 2Q illustrate the electrodes of FIGS. 2M and 2N coupled to a molded cap of the electrosurgical apparatus of FIG. 2A in accordance with an embodiment of the present disclosure;

FIGS. 2R, 2S, 2T illustrate the assembly of the distal end of the electrosurgical apparatus of FIG. 2A in accordance with an embodiment of the present disclosure;

FIG. 2U is a side cross-section view of the electrosurgical apparatus of FIG. 2A in accordance with an embodiment of the present disclosure;

FIG. 3 is a perspective view of an electrosurgical apparatus in accordance with an embodiment of the present disclosure;

FIGS. 4A and 4B illustrate an electrosurgical apparatus configured as bipolar electrosurgical forceps in accordance with an embodiment of the present disclosure;

FIGS. 4C and 4D illustrate a distal portion of the electrosurgical apparatus for FIGS. 4A and 4B in accordance with an embodiment of the present disclosure;

FIG. 5A is a partial cross-section view of the distal tip of the electrosurgical apparatus of FIG. 1A including at least one threaded connection in accordance with an embodiment of the present disclosure;

FIGS. 5B and 5C are perspective views of a connection member of the electrosurgical apparatus of FIG. 5A in accordance with an embodiment of the present disclosure;

FIG. 5D is another partial cross-section view of the distal tip of the electrosurgical apparatus of FIG. 5A in accordance with an embodiment of the present disclosure;

FIG. 5E is a perspective view of the distal tip of the electrosurgical apparatus of FIG. 5A in accordance with an embodiment of the present disclosure;

FIG. 5F is a partial cross-section view of an electrosurgical apparatus including a foam shaft in accordance with an embodiment of the present disclosure;

FIG. 5G is a partial cross-section view of the distal tip of the electrosurgical apparatus of FIG. 1A including a foam shaft and a threaded connection in accordance with an embodiment of the present disclosure; and

FIG. 5H is a partial cross-section view of another electrosurgical apparatus including a foam shaft in accordance with an embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, apparatus, applicator, handpiece, forceps, etc., which is closer to the user, while the term “distal” will refer to the end which is further from the user. Herein, the phrase “coupled” is defined to mean directly connected to or indirectly connected with through one or more intermediate components. Such intermediate components may include both hardware and software based components.

Devices and systems including one or more porous electrodes are provided. In one embodiment, an electrosurgical apparatus is provided having a shaft, a handle, and at least one porous electrode. The shaft is coupled to the handle and the at least one porous electrode is disposed on a distal tip of the shaft. The at least one porous electrode is configured to conduct electrosurgical energy provided to the distal tip and enable fluid provided to the distal tip to pass through the porous structure of the at least one electrode, such that the electrosurgical energy and the fluid are simultaneously applied to patient tissue adjacent to the at least one porous electrode. In one embodiment, the shaft is rotatable relative to the handle of the electrosurgical apparatus to change the orientation of the at least one porous electrode relative to the handle. In another embodiment, the shaft is extendable or retractable relative to the handle to increase or decrease the distance between the at least one porous electrode and the handle.

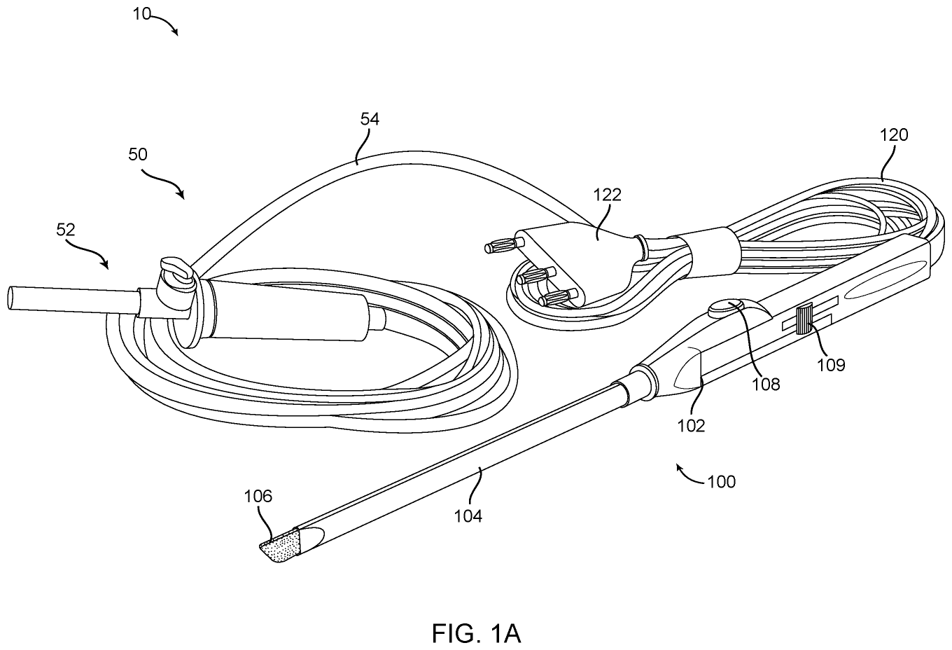

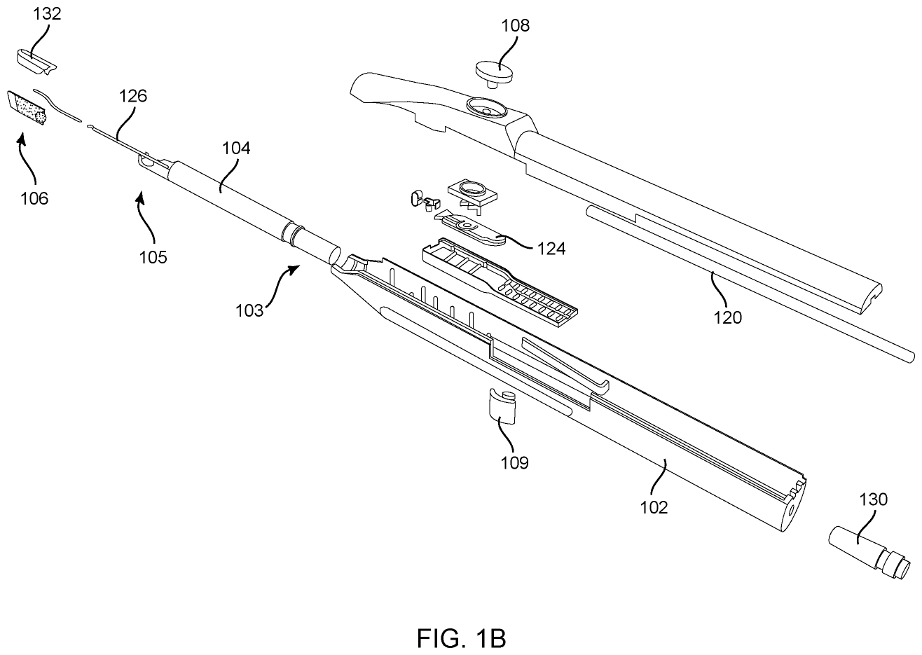

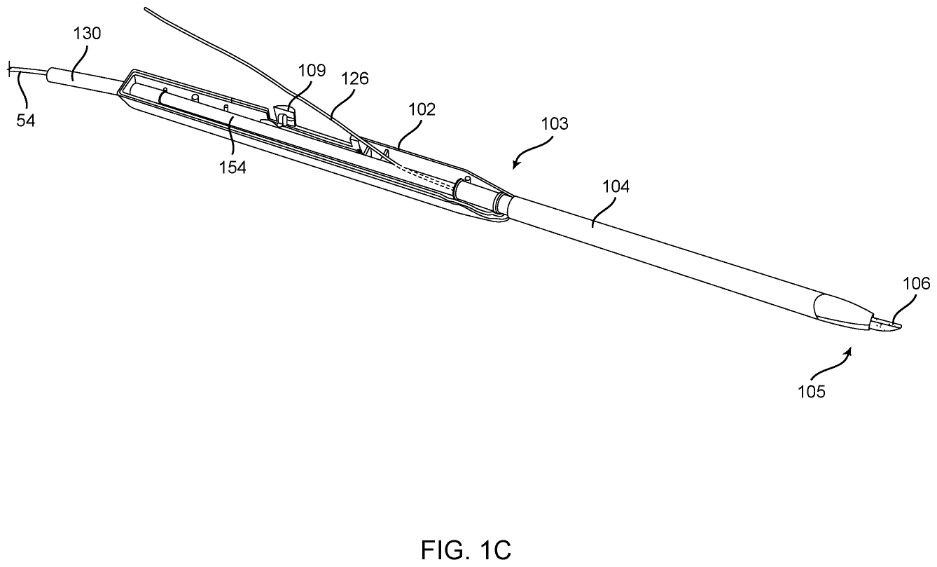

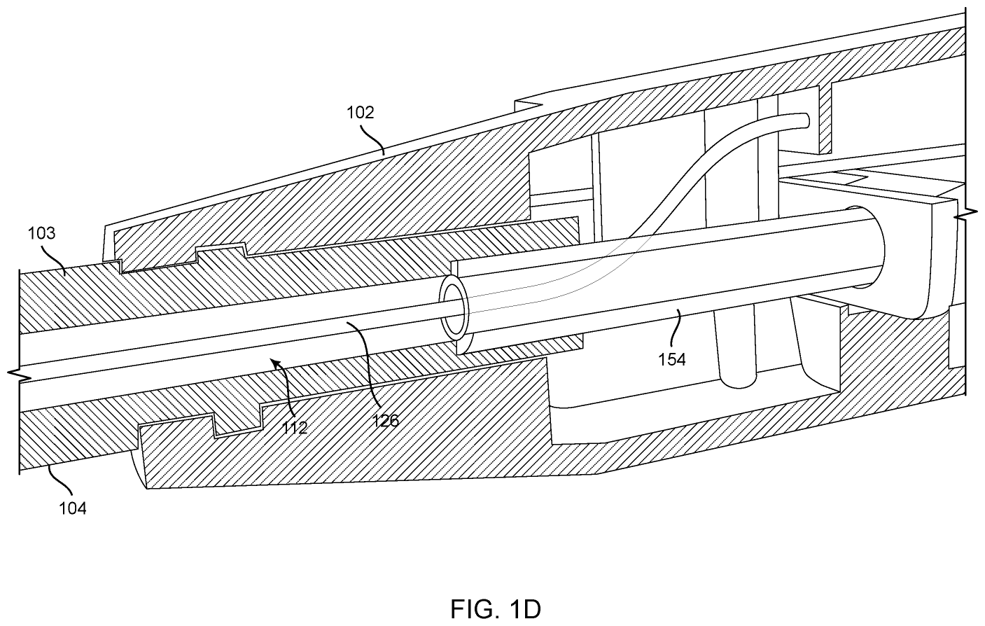

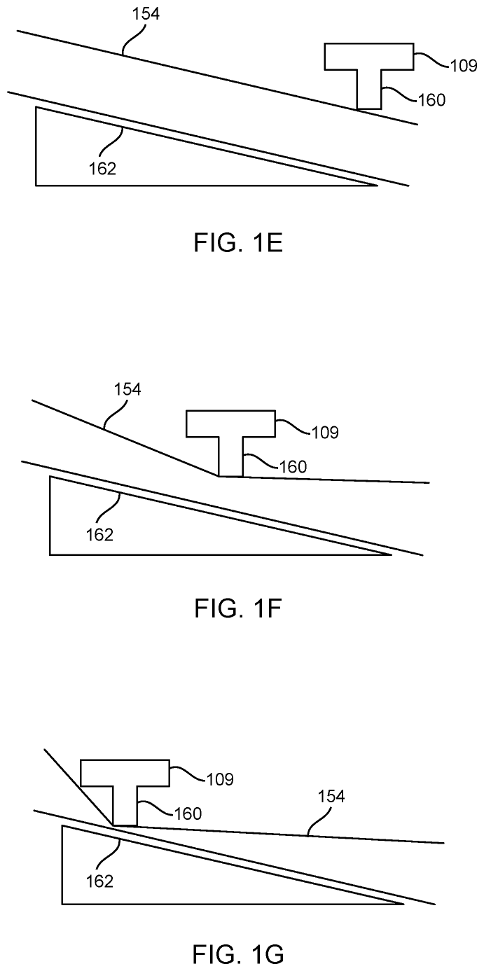

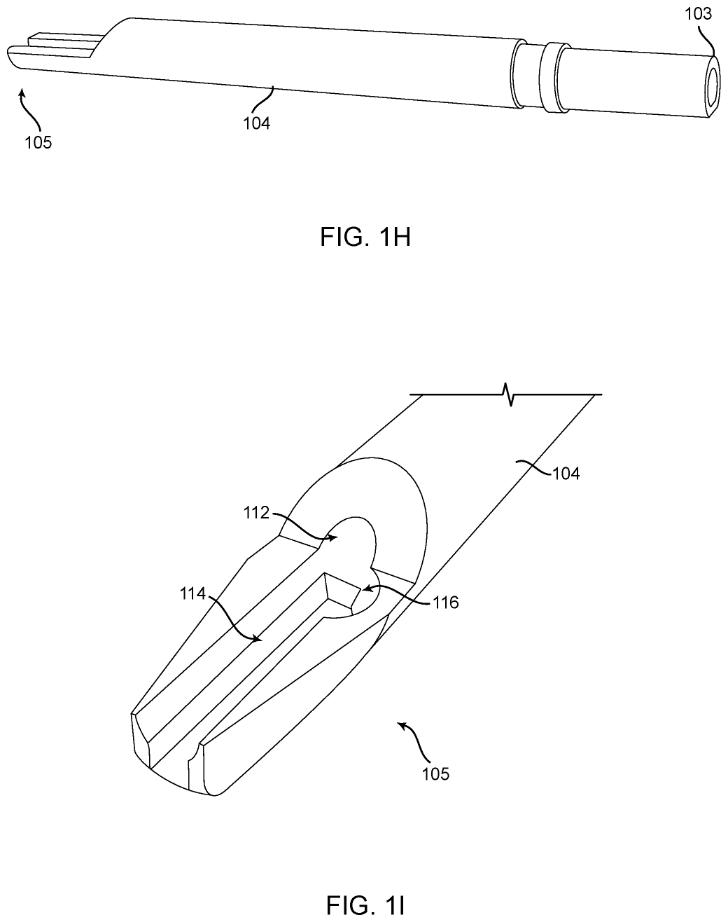

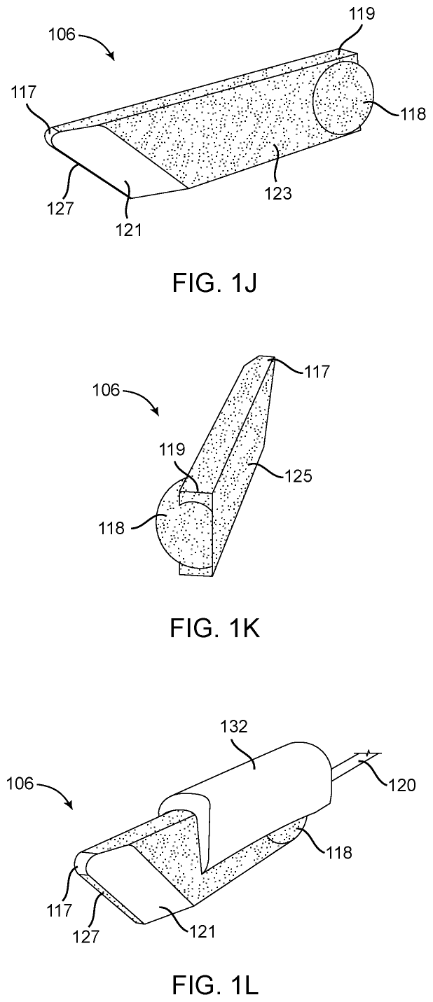

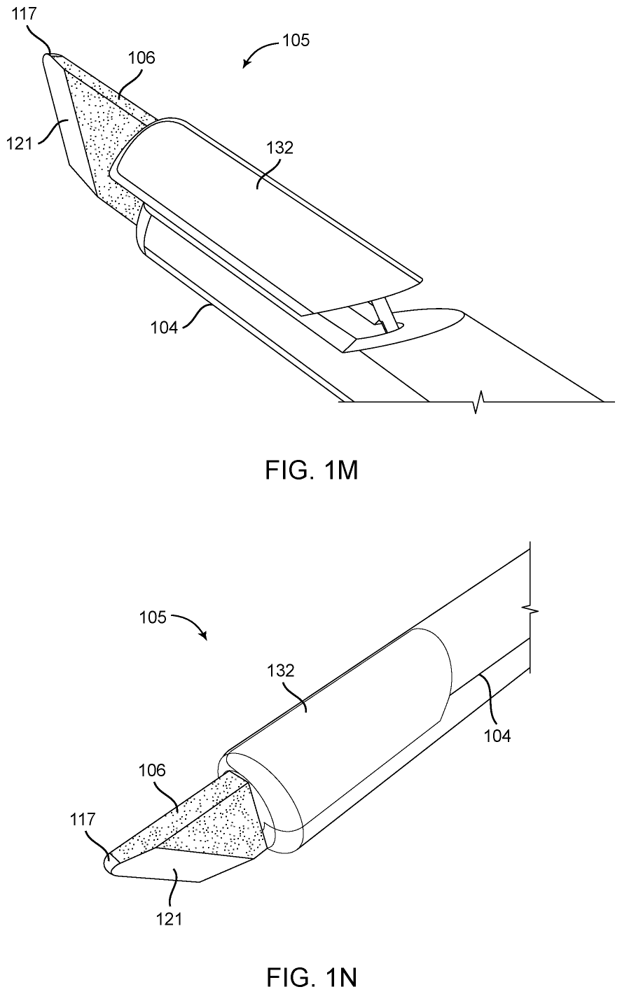

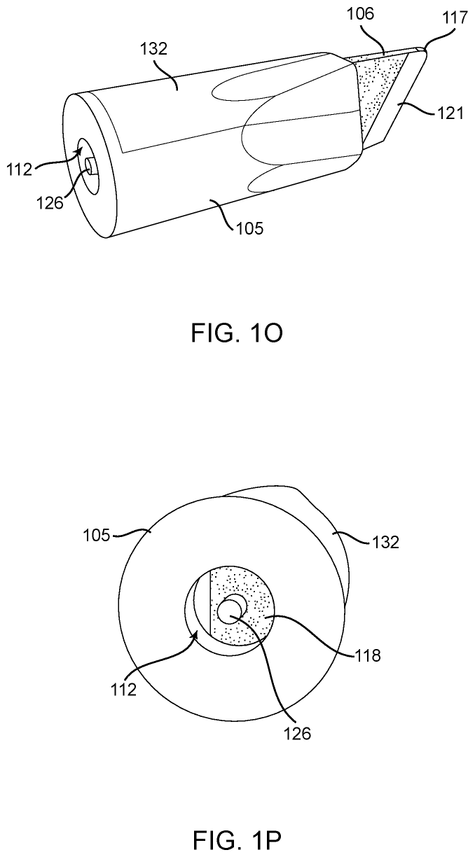

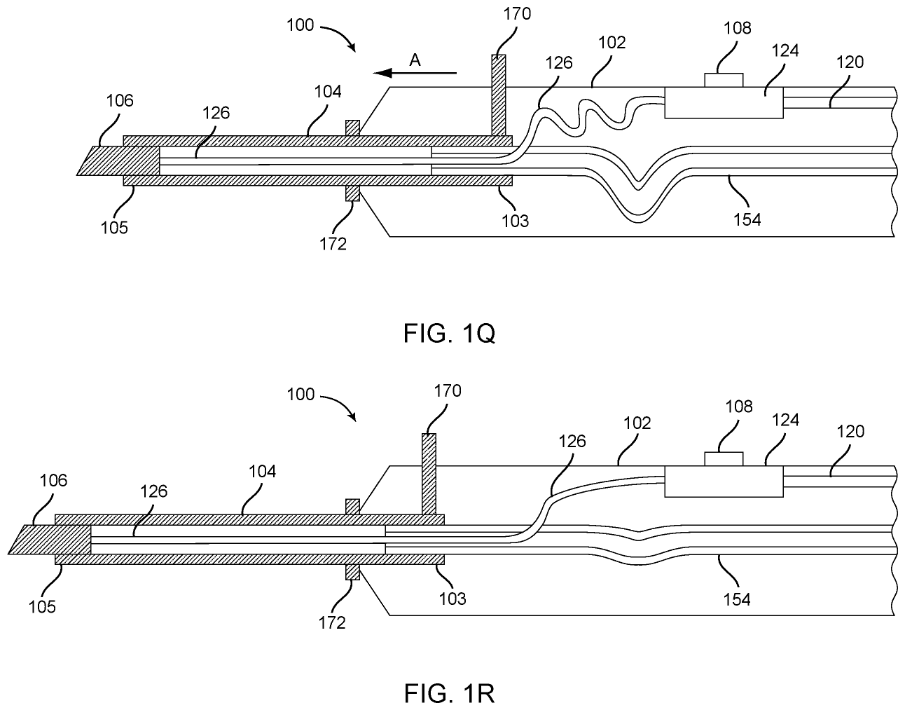

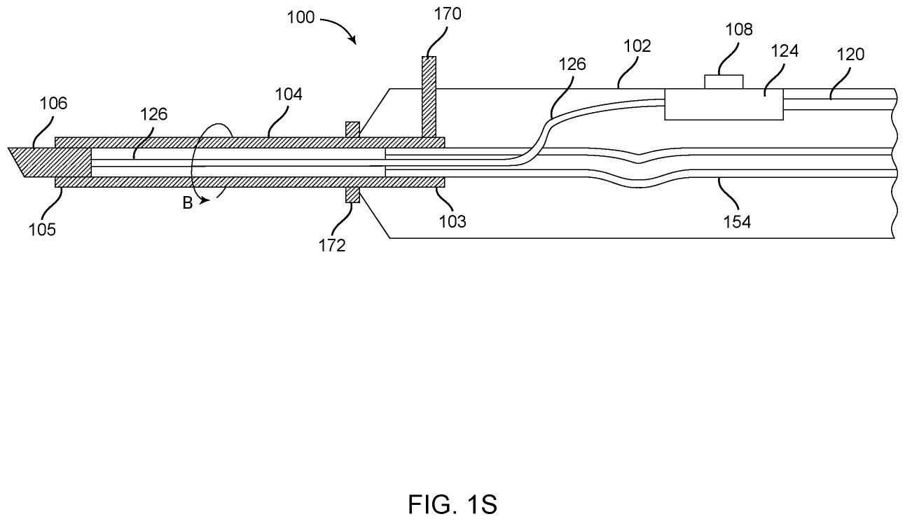

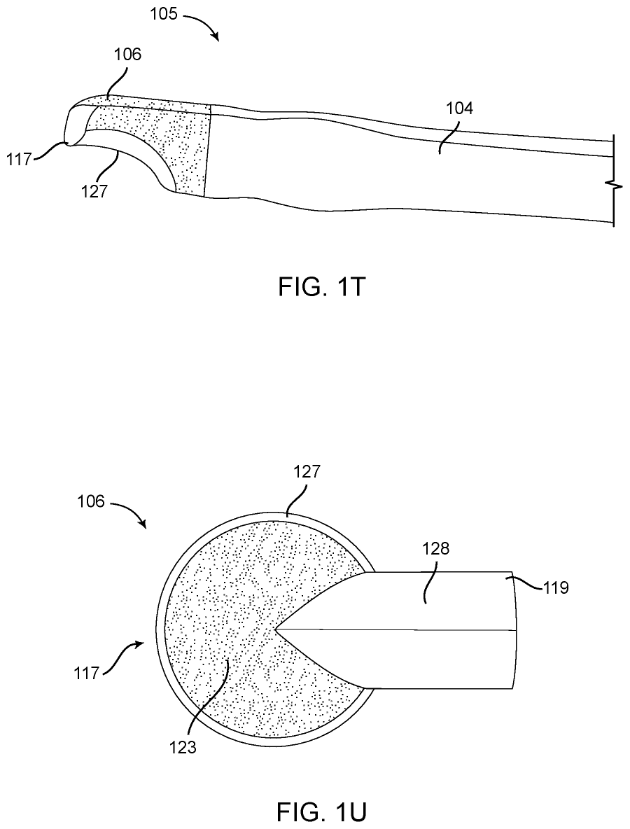

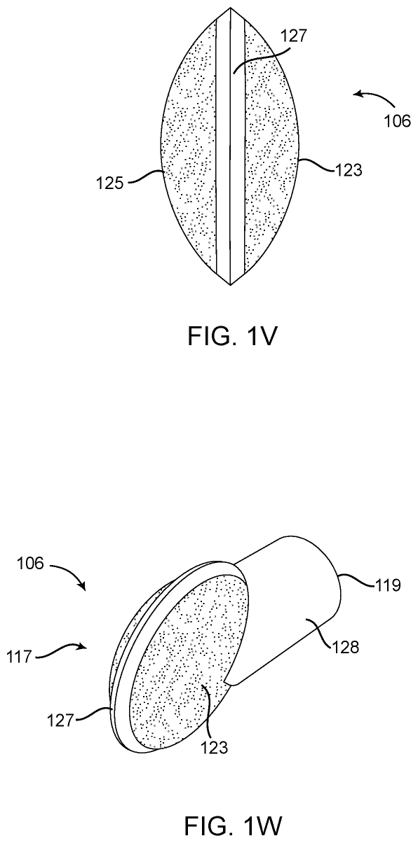

Referring to FIG. 1A, an electrosurgical system 10 is shown in accordance with the present disclosure. The system 10 of FIG. 1A, includes an electrosurgical apparatus 100 configured for performing various procedures on patient tissue (e.g., coagulation, ablation, etc.) and a fluid assembly 50 configured for providing a fluid (e.g., an electrically conducting fluid, such as saline) received from a fluid source to apparatus 100. Although not shown, the system 10 also includes an energy source (e.g., an electrosurgical generator) for providing suitable energy to apparatus 100.

Fluid assembly 50 includes a fluid gathering mechanism 52 (e.g., a syringe or other suitable mechanism) and a fluid tube 54. Mechanism 52 is configured to gather fluid from a fluid source and provide the fluid via tube 54 to apparatus 100.

Apparatus 100 includes a handle housing 102, a shaft or flow tube 104, an electrode 106, a cable 120, and a connector 122. Cable 120 couples a distal portion of housing 102 to connector 122. Connector 122 is configured to be coupled to an energy source, such that, energy (e.g., in the form of a radio frequency waveform) is provided from the energy source to apparatus 100 via one or more conductors in cable 120.

Referring to FIGS. 1B-1D, the internal components of apparatus 100 are shown in greater detail. Apparatus 100 includes a connector 130 disposed through a proximal end of handle 102 and a fluid tube 154 disposed within the interior of handle 102. A first side of connector 130 is configured to receive an end of tube 54 and a second end of connector 130 is configured to receive a proximal end of tube 154, such that tube 154 receives fluid via the fluid source assembly 50 is coupled to. A distal end of tube 154 is coupled to proximal end 103 of shaft 104 (e.g., via an adhesive of other suitable sealing method), such that fluid from the fluid source is provided to the interior of shaft 112 and to electrode 106, which is disposed in distal end 105 of shaft 104.

Cable 120 extends into the proximal end of handle 102 into the interior of handle 102 and coupled to a circuit or switch 124. Circuit 124 is further coupled to a proximal end of wire 126. Wire 126 is disposed through an outer wall of tube 154 and extends into the interior of tube 104, where a distal end of wire 126 is coupled to electrode 106. A button 108 is disposed through a wall of handle 102 and configured to contact circuit 124 to cause electrosurgical energy received from cable 120 to be applied to wire 126 and thus to electrode 106.

In one embodiment, apparatus 100 includes a flow controller or slider 109 that is configured to enable a user to control the flow rate of the fluid provided from assembly 50. The slider 109 is disposed in a slot of the handle 102, such that the slider 109 is slidable in the slot. Referring to FIGS. 1E-1G, the operation of slider 109 is shown in greater detail. In one embodiment, the interior of handle 102 includes a surface 162, which is inclined with respect to the path of motion of slider 109 through the slot. Slider 109 includes an extension member 160. A portion of tube 154 passes through a gap between extension member 160 and surface 162 within handle 102. Extension member 160 is configured to constrict tube 154 between extension member 160 and surface 162 as slider 109 is advanced through the slot. By selectively constricting the tube 154, the diameter of tube 154 can be changed as desired by a user to change the flow rate of the fluid passing through tube 154. For example, when slider 109 is at a first end of the slot, as shown in FIG. 1E, the extension member 160 is disposed at a sufficiently great distance from surface 162, such that tube 154 is not constricted and the flow rate of a fluid through tube 154 is at maximum. When slider 109 is advanced through the slot from the first position, as shown in FIG. 1F, the distance between the extension member 160 and the surface 162 lessens and the extension member 160 constricts tube 154, thus reducing the diameter of tube 154 and the allowable flow rate of a fluid passing through tube 154. When slider 109 is advanced to reach a second end of the slot (e.g., opposite to the first end), the distance between extension member 160 and surface 162 becomes sufficiently small such that the diameter of tube 154 does not permit any fluid to flow within tube 154 toward shaft 104.

In another embodiment, slider 109 (or, alternatively, another button or control on handle 102) is configured to control the flow rate of the fluid provided from assembly 50 by providing a control signal to assembly 50 when slider 109 is engaged by a user. In this embodiment, assembly 50 includes a pump for providing the fluid to apparatus 100 via tube 54. The pump in assembly 50 (or a controller or processor controlling the pump) is electrically coupled (e.g., via a wire) to a switch (e.g., switch 124 or a separate switch) of apparatus 100, where the switch is further electrically coupled to slider 109. When a user engages slider 109, the switch generates a control signal based on the position of slider 109 that is provided to the pump (or a controller or processor controlling the pump) to selectively control the flow rate that the fluid is provided to apparatus 100 via tube 54.

Referring to FIGS. 1H and 1I, shaft 104 is shown in greater detail. Shaft 104 includes a channel 112 extending from proximal end 103 to distal end 105. Distal end 105 includes a slot 114 configured to receive electrode 106. A portion 116 of slot 114 is configured with a semi-spherical slot or groove 116. Referring to FIGS. 1J and 1K, in one embodiment, electrode 106 is configured in a substantially planar shape and as a blade. Electrode 106 extends from a proximal end 119 to distal end 117 and includes sides 123, 125. End 117 is configured with a tapered point and a beveled surface 121. Side 125 of electrode 106 is flat. A hemispherical protrusion 118 extends away from side 123. It is to be appreciated that although slot 116 is shown in a semi-spherical shape and protrusion 118 is shown in a hemispherical shape, in other embodiments, protrusion 118 and slot 116 may be configured in any other shapes having a keyed relationship. For example, in other embodiments, protrusion 118 is configured in a rectangular, triangular, or irregular shape extending away from side 123 and slot 116 is configured in a suitable shape for receiving protrusion 118.

Electrode 106 is received by distal end 105 of shaft 104, such that a portion of electrode 106 is disposed in slot 114 and hemispherical protrusion 118 is disposed in semi-spherical slot 116. Referring to FIG. 1I, 1M, 1L, 1N, to secure electrode 106 to distal end 105 of shaft 104, a cap 132 is coupled to proximal end 119 of electrode 106. Cap 132 includes a slot (not shown) for receiving a portion of electrode 106. In one embodiment, electrode 106 is coupled to the distal end 105 of shaft 104 via injection molding. For example, in this embodiment, cap 132 is injection molded (e.g., using a thermoplastic or other suitable material) over proximal end 119 of electrode 106 to couple electrode 106 to distal end 105. In another embodiment, cap 132 is bonded to distal end 105 of shaft 104 (e.g., using a suitable adhesive substance) to couple electrode 106 to end 105. Since protrusion 118 is disposed in slot 116, electrode 106 is prevented from becoming separated or severed from shaft 104 while apparatus 100 is in use.

Referring FIGS. 1O and 1P, cross-section views of distal end 105 of shaft 104 with electrode 106 coupled to shaft 104 using cap 132 are shown in accordance with the present disclosure. As shown in FIGS. 1O-1P, wire 126 is disposed through channel 112 and coupled to hemisphere protrusion 118 on the proximal end of electrode 106. Channel 112 is configured with a sufficiently large diameter to ensure that the fluid flow through channel 112 is not undesirably restricted and enables adequate fluid flow. It is to be appreciated that distal end 105 of shaft 104 may be sealed in a variety of ways to be prevent fluid leakage between electrode 106 and distal end 105. For example, in one embodiment, a shrink wrap material may be disposed over a distal end 105 (i.e., including cap 132) of shaft 104 and a proximal portion of electrode 106 to prevent fluid leakage. In another embodiment, suitable adhesives may be used to coupled electrode 106 to distal end 105 to seal the connection between distal end 105 and electrode 106 and prevent fluid leakage.

In use, shaft 104 of apparatus 100 optionally may be disposed through a cannula or trocar and into a tissue structure of a patient to perform an electrosurgical procedure on patient tissue. When slider 109 is in a suitable position, fluid (e.g., saline) received from a fluid source is provided via channel 112 to electrode 106. When button 108 is pressed, electrosurgical energy received from an electrosurgical generator coupled to apparatus 100 is provided via wire 126 to energize electrode 106, such that an electrosurgical effect (e.g., cutting or coagulation) is realized. It is to be appreciated that the electrosurgical generator coupled to apparatus 100 may be configured with various waveforms configured to provide differing tissue effects when applied to electrode 106. In one embodiment, one or more controls (e.g., buttons, sliders, etc.) may be provided on handle 102 and coupled to circuit or switch 124. The one or more controls are configured to enable a user to select different waveforms to be applied by the electrosurgical generate to electrode 106. When the one or more controls are engaged by a user, a control signal is generated by switch 124 and provided to the electrosurgical generator via cable 120 to selectively apply different waveforms to electrode 106.

Electrode 106 is made of a conductive material (e.g., stainless steel) having a porous structure that renders the electrode 106 pervious to the passage of fluid, thereby facilitating the uniform distribution of an electrically conductive fluid into the tissue during the ablation process. The porous structure allows fluid to not only pass through the electrode 106. In addition to providing a more uniform distribution of fluid, the porous structure of electrode 106 is configured such that tissue is less apt to stick to the surfaces of the electrode 106 while electrode 106 is in use and a fluid, such as saline, is provided through the pores of electrode 106.

To this end, the porous structure of electrode 106 comprises a plurality of pores that are in fluid communication with channel 112 of shaft 104. In one embodiment, the pores of the porous structure are configured to be interconnected in a random, tortuous, interstitial arrangement in order to maximize the porosity of the electrodes 106. The porous structure may be microporous, in which case, the effective diameters of the pores are in the 0.05-20 micron range, or the porous structure may be macroporous, in which case, the effective diameters of the pores are in the 20-2000 micron range. In one embodiment, the pore size will be in the 1-50 micron range. The porosity of the porous structure, as defined by the pore volume over the total volume of the structure, may be in the 20-80 percent range. Naturally, the higher the porosity, the more freely the fluid will flow through the electrodes 106. Thus, the designed porosity of the porous structure will ultimately depend on the desired flow of the fluid through electrode 106.

Thus, it can be appreciated that the pervasiveness of the pores in the porous structure enables the fluid to freely flow from channel 112, through the thickness of the electrode 106, and out to the tissue adjacent to electrode 106. It is to be appreciated that this free flow of fluid occurs even if several of the pores have been clogged with material, such as tissue. In one embodiment, the porous structure provides for the wicking (i.e., absorption of fluid by capillary action) of fluid into the pores of the porous structure. To promote the wicking of fluid into the porous structure, the porous structure may be hydrophilic.

In some embodiments, electrode 106 is configured such that two or more selected regions of electrode 106 include or are configured with varying or differing levels or amounts of porosity relative to each other. The varying porosity in the selected regions are configured such that more or less fluid may be delivered through certain regions of the porous structure as desired. In some embodiments, certain regions are configured with zero porosity (e.g., having no interconnected channels), such that, no fluid may be pass through these regions. In this way, the path the fluid takes through porous structure of electrode 106 to escape may be controlled.

In some embodiments, it may be advantageous to configure the sharp edges of an electrode to be solid (e.g., having zero porosity) and configure the body of the electrode to be porous. For example, referring to FIG. 1J, in one embodiment, electrode 106 includes a beveled surface 121 forming a sharp edge 127. Beveled surface 121 is configured to be solid (e.g., having zero porosity), while the remaining portions of electrode 106 are configured with a porous structure. Thus, no fluid passes through beveled surface 121 and edge 127, instead fluid is directed to exit via the remaining regions or portions of electrode 106 having a higher, non-zero porosity than surface 121 and edge 127. The solid surface 121 and edge 127 (e.g., zero porosity) enable a higher energy density to develop on surface 121 and edge 127 when electrode 106 is energized to support electrosurgical cutting. Furthermore, the beveled surface 121 and sharp edge 127 are configured to support mechanical cutting when electrode 106 is de-energized. It is to be appreciated that, in some embodiments, electrode 106 may be configured with at least one second beveled surface forming at least one second sharp edge. The at least one second beveled surface may be configured to be solid (e.g., zero porosity).

The porous structure preferably is composed of a metallic material, such as stainless steel, titanium, or nickel-chrome. While electrode 106 is preferably composed of an electrically conductive material, the electrode 106 may alternatively be composed of a non-metallic material, such as porous polymer or ceramic. While the porous polymers and ceramics are generally non-conductive, they may be used to conduct electrical energy to the tissue by virtue of the conductive fluid within the interconnected pores of electrode 106.

In some embodiments, the porous structure may be made of a mix of materials having various degrees of conductivity. For example, certain portions or regions of the porous structure may be made of a conductive material, while other portions or regions are made of a non-conductive (or less conductive) material to selectively control the path of delivery of electrosurgical energy through electrode 106.

In one embodiment, the porous structure is formed using a sintering process, which involves compacting a plurality of particles (preferably, a blend of finely pulverized metal powers mixed with lubricants and/or alloying elements) into the shape of the electrode 106, and then subjecting the blend to high temperatures. When compacting the particles, a controlled amount of the mixed powder is automatically gravity-fed into a precision die and is compacted, usually at room temperature at pressures as low as 10 or as high as 60 or more tons/inch2 (138 to 827 MPa), depending on the desired porosity of the electrode 106. The compacted powder will have the shape of the electrode 106 once it is ejected from the die, and will be sufficiently rigid to permit in-process handling and transport to a sintering furnace. Other specialized compacting and alternative forming methods can be used, such as powder forging, isostatic pressing, extrusion, injection molding, spray forming, and/or three-dimensional (3D) printing.

During sintering, the unfinished electrode 106 is placed within a controlled-atmosphere furnace, and is heated to below the melting point of the base metal, held at the sintering temperature, and then cooled. The sintering transforms the compacted mechanical bonds between the powder particles to metallurgical bonds. The interstitial spaces between the points of contact will be preserved as pores. The amount and characteristics of the porosity of the structure can be controlled through powder characteristics, powder composition, and the compaction and sintering process.

It is to be appreciated that porous structures can be made by methods other than sintering. For example, pores may be introduced by mechanical perforation, by the introduction of pore producing agents during a matrix forming process, or through various phase separate techniques. The porous structure may be made by 3D printing. Also, the porous structure may be composed of a ceramic porous material with a conductive coating deposited onto the surface, e.g., by using ion beam deposition or sputtering.

The usage of a conductive material including a porous structure for electrode 106 enables a fluid, such as saline, to be provided to the tissue concurrently with the electrosurgical energy applied via electrode 106 to the patient tissue. This effect has many benefits, including, but not limited to, (1) faster, but controlled, dissection, (2) less charring of tissue, (3) electrode 106 remains cleaner during use (i.e., less tissue sticks to electrode 106, resulting in less re-bleeding when electrode 106 is pulled off tissue), (4) less smoke is produced from heated tissue, (5) greater depth of coagulation is realized, and (5) vessel sealing occurs.

Referring to FIGS. 1Q and 1R, in one embodiment, apparatus 100 is configured such that shaft 104 is extendable distally or retractable proximally relative to handle 102. In this embodiment, shaft 104 is coupled to handle 102, such that shaft 104 is extendable or retractable relative to handle 102. A slider 170 is disposed through a slot in the outer wall of handle 102 and coupled to a proximal portion of the exterior of shaft 104. By advancing slider 170 distally relative to handle 102 (as denoted by the letter A in FIG. 1Q), shaft 104 and electrode 106 are also advanced distally relative to handle 102 (as shown in FIG. 1R). Alternatively, by retracting slider 170 proximally relative to handle 102 (e.g., in a direction opposite to direction A), shaft 104 and electrode 106 are also retracted proximally relative to handle 102 (as shown in FIG. 1Q). It is to be appreciated that in this embodiment, wire 126 and fluid tube 154 are configured to be flexible and with a sufficient amount of slack to accommodate the distal extension and proximal retraction of shaft 104 relative to handle 102. In another embodiment, fluid tube 154 may be configured with a telescoping mechanism (e.g., one or more folds in a portion of the outer wall of tube 154) such that the length of tube 154 may be lengthened or shortened as necessary to accommodate the distal extension and proximal retraction of shaft 104 relative to handle 102. The embodiment of FIGS. 1Q and 1R advantageously enable a user to change the length of shaft 104, thus enabling apparatus 100 to be used for electrosurgical procedures requiring electrode 106 to require various depths within the cavity of the patient that shaft 104 is disposed through.

Referring to FIG. 1S, in one embodiment, shaft 104 may be mounted to handle 102 such that shaft 104 is rotatable relative to handle 102 (e.g., as denoted by the arrow labelled with the letter B in FIG. 1S) to change the orientation of electrode 106 (e.g., compare the orientation of electrode 106 in FIGS. 1Q and 1R to the orientation of electrode 106 in FIG. 1S). In this embodiment, apparatus 100 further includes a knob or rotation actuation member 172 disposed around the exterior of shaft 104 and exterior to the handle 102. Knob 172 is configured such that when knob 172 is rotated with respect to handle 102, shaft 104 is also rotated with respect to handle 102, thus enabling a user to selectively change the orientation of electrode 106 with respect to handle 102 without requiring a user to rotate handle 102.

In one embodiment, shaft 104 is configured to be rotatable relative to flow tube 154. In this embodiment, a seal is disposed between shaft 104 and flow tube 154 to enable shaft 104 and tube 154 to be rotatable relative to each other without leaking fluid (e.g., saline) provided through tube 154 and shaft 104.

Apparatus 100 is configured for use in open or laparoscopic surgical procedures. In one embodiment, where apparatus 100 is used for laparoscopic surgical procedures, handle 102 is configured in the shape of a pistol having a pistol grip with controls (e.g., button 108, slider 110, slider 170, knob 172, etc.) placed on and/or proximately to the pistol grip to enable convenient control for the user.

It is to be appreciated that in the embodiments described above, shaft 104 is configured to be rigid and linear. However, in other embodiments of the present disclosure, shaft 104 may be configured to be flexible to enable shaft 104 to be bent such that distal end 105 of shaft 104 may achieve a variety of different orientations with respect to handle 102. In some embodiments, shaft 104 may be flexible and distal end 105 may be configured to be grasped by forceps of a robotic arm to manipulate the orientation of distal end 105 of shaft 104.

Furthermore, it is to be appreciated that, in other embodiments, electrode 106 may be configured in different geometries than shown in FIGS. 1J, 1K, 1L. For example, referring to FIG. 1T, in one embodiment, edge 127 of electrode 106 may be configured in a concave shape (e.g., a hook shape). As another example, electrode 106 may be configured in a ball shape.

As another example, referring to FIGS. 1U, 1V, 1W, a distal portion of electrode 106 may be configured with convex sides 123, 125, which tapper to and share a convexly curved sharp edge 127. The proximal portion of electrode 106 is configured as a base 128 that is received by distal end 105 of shaft 104. As described above, in some embodiments, one or more regions of electrode 106 may be configured with varying degrees of porosity to control the flow of fluid through electrode 106. In the embodiment shown in FIGS. 1U-1W, base 128 and edge 127 are configured to be solid and with a lower porosity (e.g., essentially zero porosity) than the remaining regions of electrode 106. Thus, when no fluid passes through base 128 and edge 127. Sides 123, 125 are each configured with porous structures, thus enabling fluid provided to electrode 106 to escape electrode 106 via sides 123, 125. The solid edge 127, being configured with a lower porosity than sides 123, 125, enables a higher energy density to develop on edge 127 when electrode 106 is energized to support electrosurgical cutting. Furthermore, the tapered edge 127 is sharp to support mechanical cutting when electrode 106 is de-energized. A shrink wrap may be disposed over base 128 and distal end 105 of shaft 104 to seal the connection between electrode 106 and shaft 104 and prevent fluid leakage at the connection. Alternatively, a suitable adhesive material may be used to seal and bond base 128 to a receiving portion in end 105 of shaft 104. It is to be appreciated that, since base 128 is configured to be solid (e.g., having zero porosity), fluid leakage is further reduced as no fluid escapes from base 128.

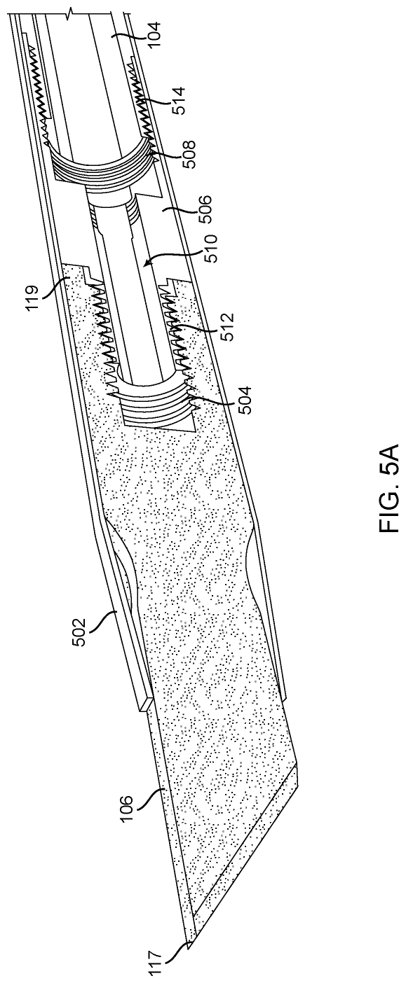

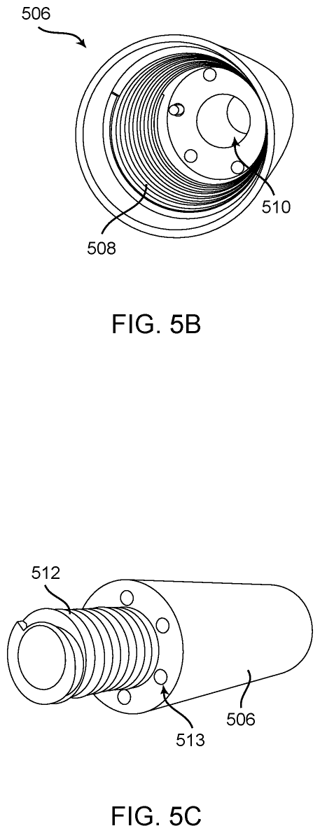

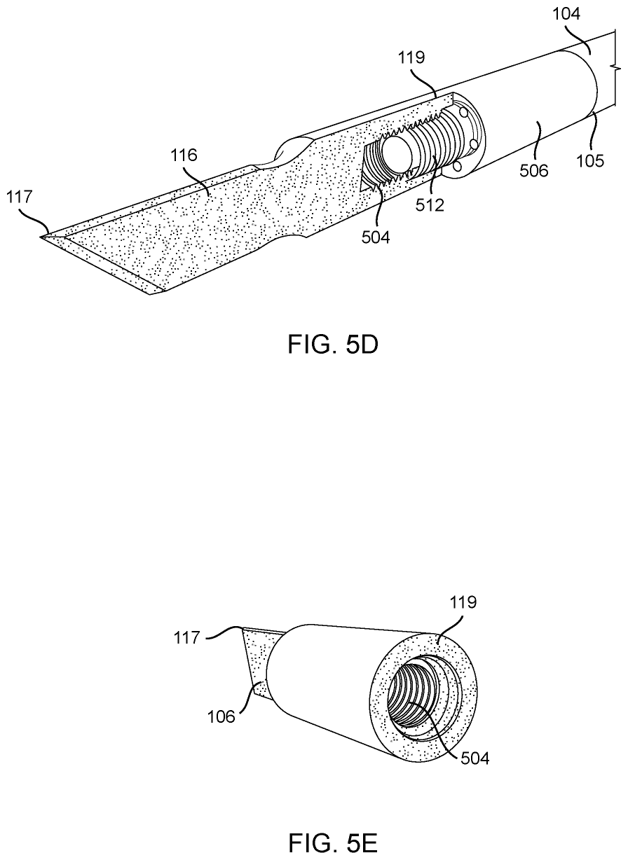

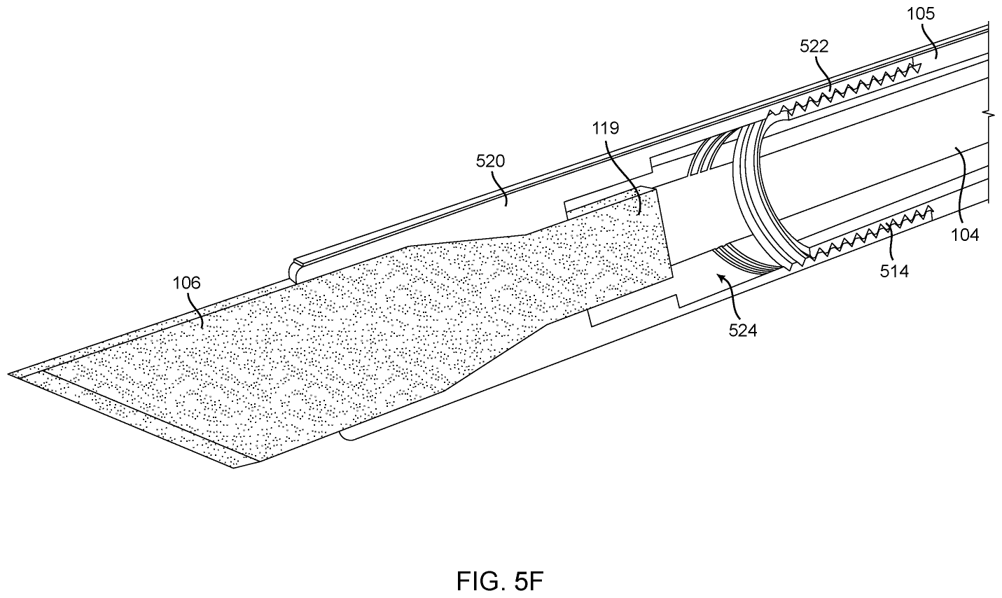

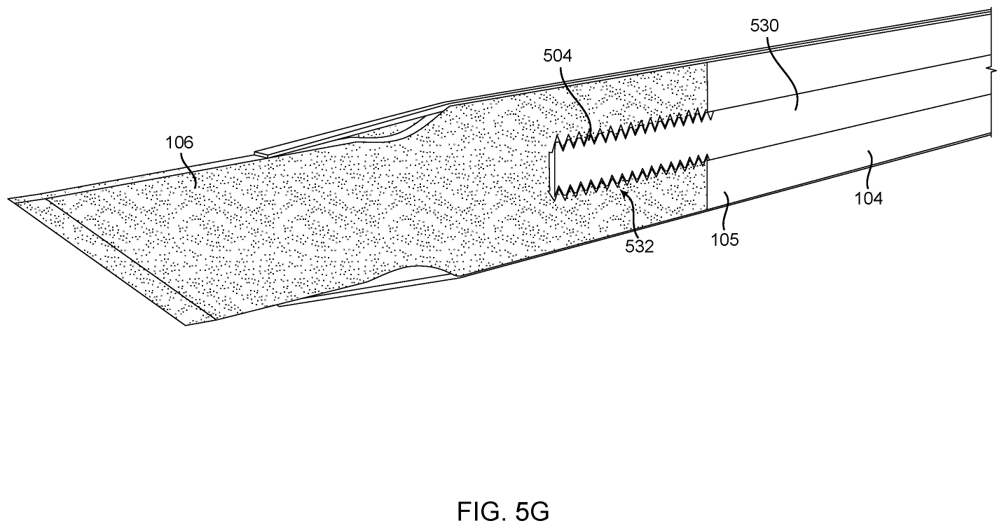

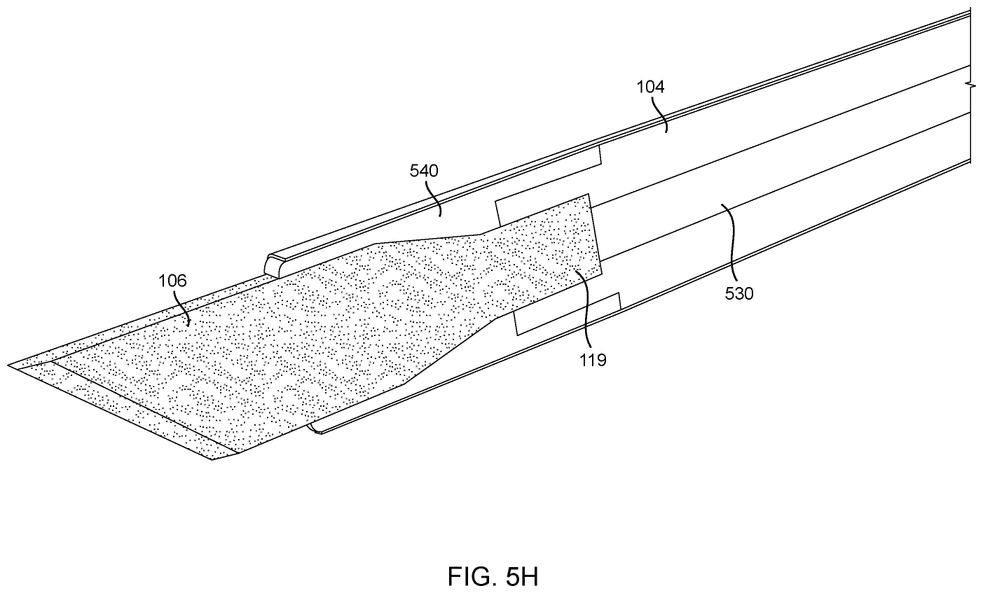

Referring to FIGS. 5A-5E, in another embodiment of the present disclosure, electrode 106 may be coupled to distal end 105 of shaft 104 via a threaded connection. For example, in the embodiment shown in FIGS. 5A-5E, proximal end 119 of electrode 106 includes female threads 504. In this embodiment, shaft 104 is made of a conducting material (e.g., stainless steel) and a proximal end of shaft 104 is coupled via a conductor to circuit 124 and cable 120 to receive electrosurgical energy. The distal end 105 of shaft 104 includes male threads 514. Apparatus 100 further includes a threaded connection member 506 made of a conductive material (e.g., stainless steel). Member 506 includes female threads 508 disposed on a proximal portion of member 506 and male threads 512 disposed on a distal portion of member 506. A channel 510 extends through the interior from the proximal and to the distal end of member 506. To couple electrode 106 to shaft 104, the distal end 105 of shaft 104 is received by the proximal end of member 506 and the male threads 514 of shaft 104 are mated with the female threads of member 506. Furthermore, the distal end of member 506 is received by the proximal end 119 of electrode 106 and the male threads 512 of member 506 are mated with the female threads 504 of electrode 106. Electrosurgical energy is provided via shaft 104 and member 506 to electrode 106. Furthermore, a fluid (e.g., saline) is provided via the interior of shaft 104 and channel 510 to electrode 106. The exteriors of shaft 104 and member 506 and a proximal portion of electrode 106 are covered by an insulative material 502.

In one embodiment, member 506 includes a plurality of apertures 513 configured to enhance the fluid flow from shaft 104 to electrode 106. In another embodiment, member 506 is integrated into the distal end 105 of shaft 104 as single shaft component.

Referring to FIG. 5F, in another embodiment, electrode 106 may be coupled to shaft 104 via a plastic threaded connection member 520. In this embodiment, shaft 104 is made of a conductive material (e.g., stainless steel) and member 520 includes a hollow interior 524 extending from the distal end to the proximal end of member 520. The hollow interior 524 includes female threads 522, which are disposed toward the proximal end of member 520. To couple electrode 106 to shaft 104, the distal end of shaft 104 is inserted into the proximal end of member 520 and male threads 514 are mated with female threads 522. The distal end 119 (which, in this embodiment, does not include male threads) is inserted into the distal end of member 520. Fluid (e.g., saline) is provided via shaft 104 and interior 524 to electrode 106. Although not shown, distal end 119 of electrode 106 is electrically coupled to shaft 104 via at least one conductor. In one embodiment, the conductor may be an insulated wire extending through and embedded in a wall of member 520. In another embodiment, shaft 104 may be made of a non-conducting material and the conductor may extend through the interior or through the outer shaft wall of shaft 104 to couple electrode 106 to circuit or switch 124. In another embodiment, electrode 106, member 520, and/or shaft 104 may be bonded to each other via ultrasonic welding or other bonding means. Shaft 104 is coupled to an electrosurgical generator (e.g., via circuit 124 and cable 120) for providing electrosurgical energy to electrode 106 received via the electrosurgical generator.

In another embodiment, shaft 104 may be made of or includes a foam material configured to enable a fluid (e.g., saline) to flow through the foam material to electrode 106. For example, referring to FIG. 5G, shaft 104 is shown made of a foam material having interconnected channels configured to allow a fluid to flow through the foam material from the proximal end 103 of shaft 104 to the distal end 105 of shaft 104. In this embodiment, the structural component of the shaft 104 (i.e., the foam material) is also used to conduct fluid flow through shaft 104. In this embodiment, apparatus 100 further includes a conductor 530 (e.g., a rod or wire) having a male threads 532 disposed on a distal end of conductor 530. Conductor 530 is disposed through the foam material of shaft 104 and extends along the length of shaft 104. The distal end of conductor 530 is disposed through the proximal end of electrode 106 and male threads 532 of conductor 530 are mated (e.g., screwed on to) with female threads 504 of electrode 106 to electrically and mechanically connect electrode 106 to conductor 530. The proximal end of conductor 530 is coupled to circuit 124 for providing electrosurgical energy to electrode 106.

In another embodiment of the present disclosure, the shaft 104 (including the foam material) and conductor 530 of FIG. 5G may be coupled to electrode 106 via an over-molded tip instead of a threaded connection. For example, referring to FIG. 5H, apparatus 100 includes an over-molded tip 540 configured to couple the distal ends of shaft 104 and conductor 530 to the proximal end of electrode 106. Tip 540 is molded (e.g., via injection molding a thermoplastic material) over a proximal portion of electrode 106 and a distal portion (e.g., including a stepped-end 107) of shaft 104 to couple shaft 104 to electrode 106. A distal end of conductor 530 is electrically coupled to electrode 106.

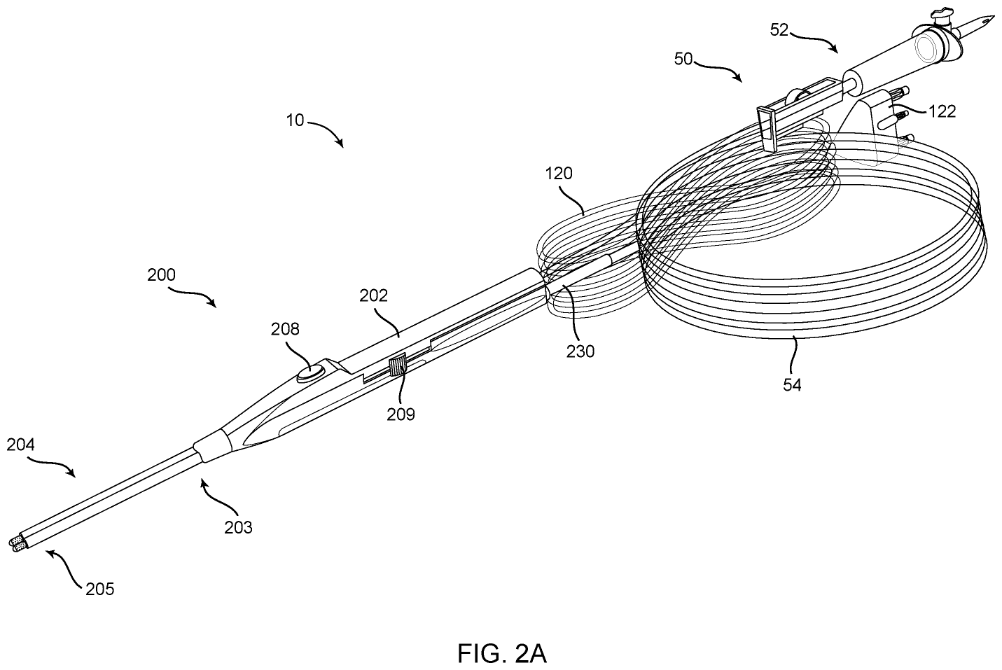

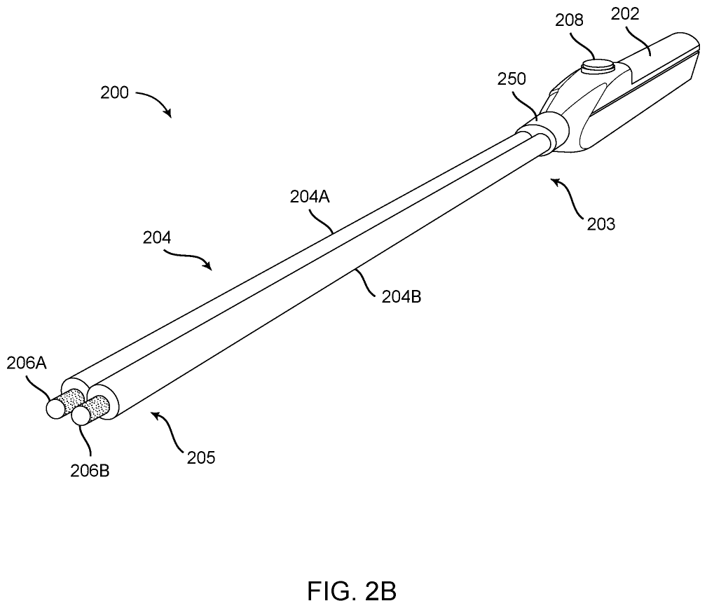

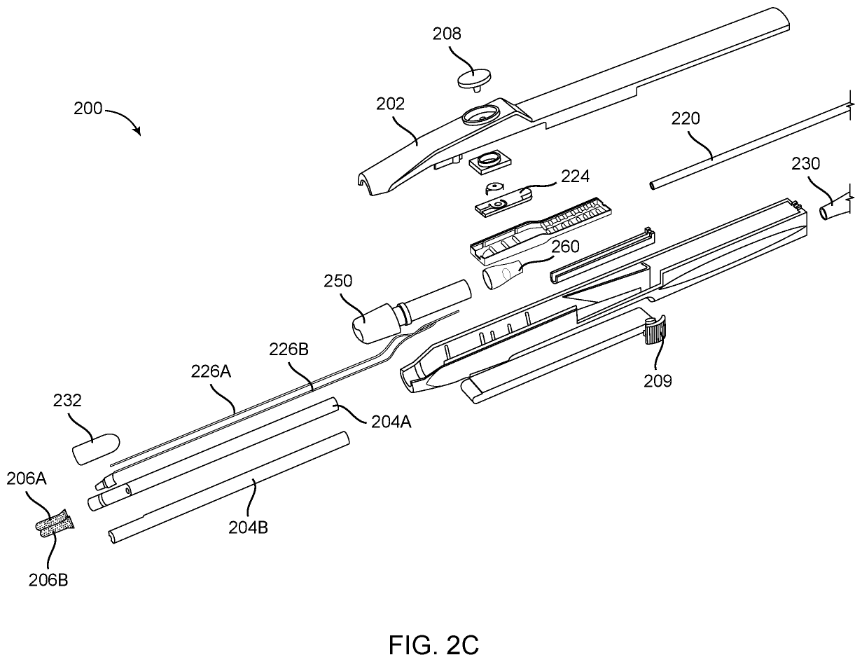

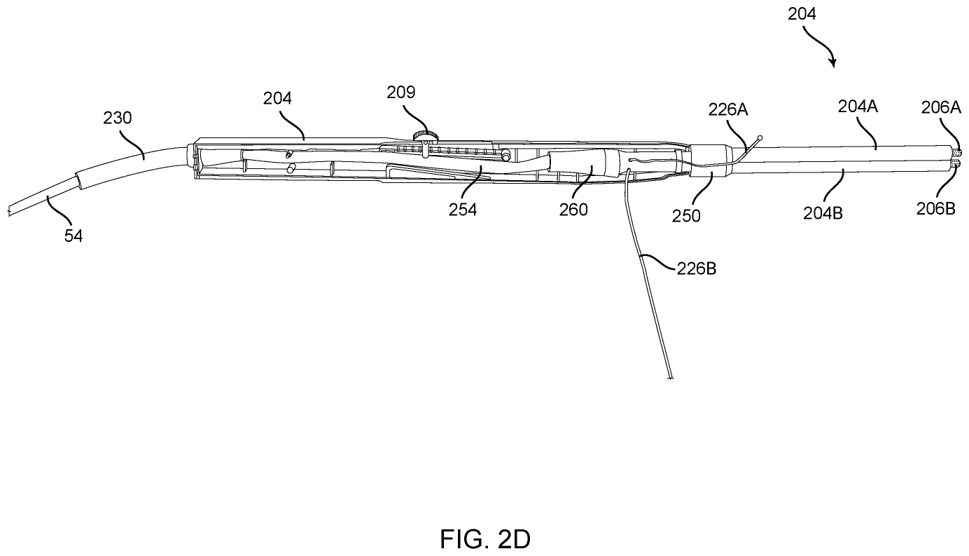

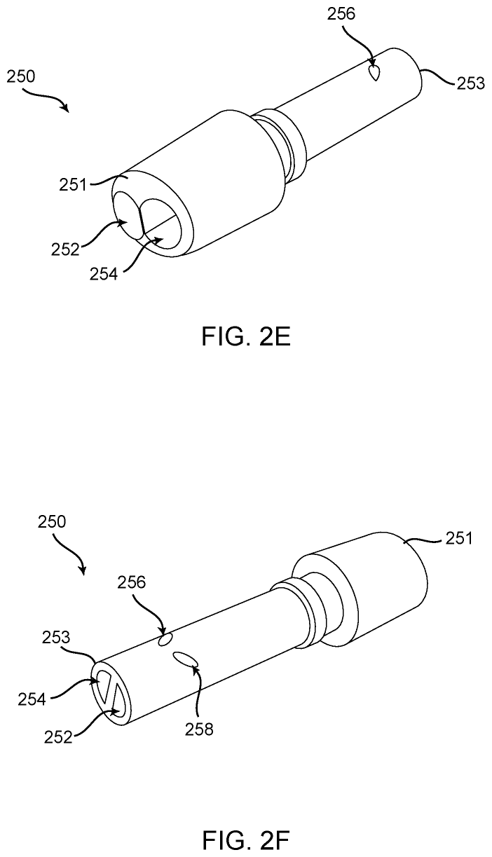

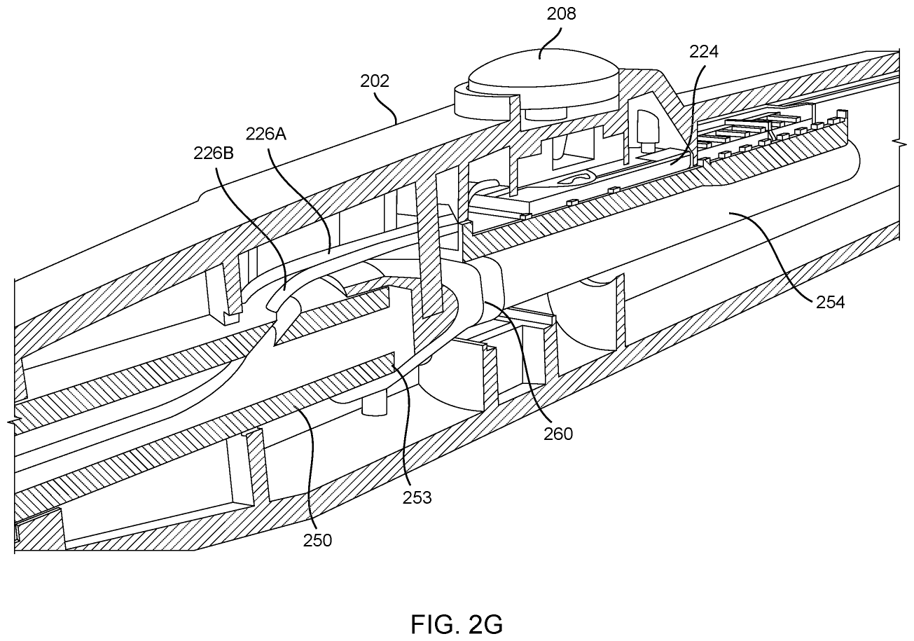

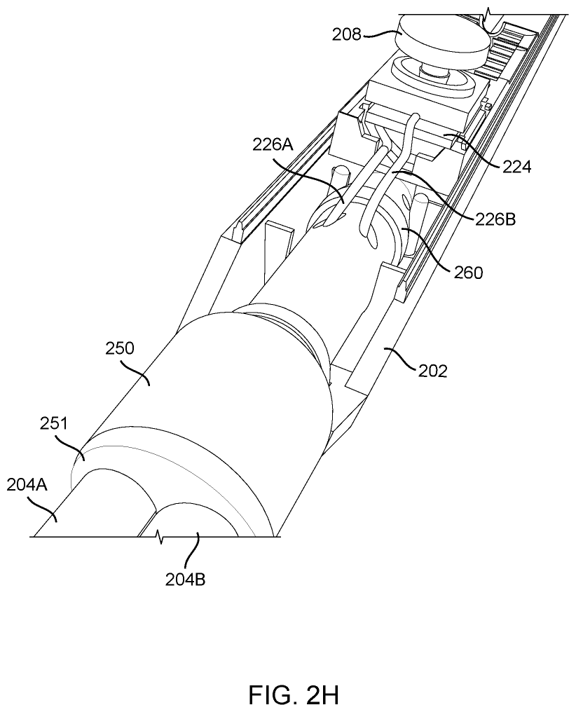

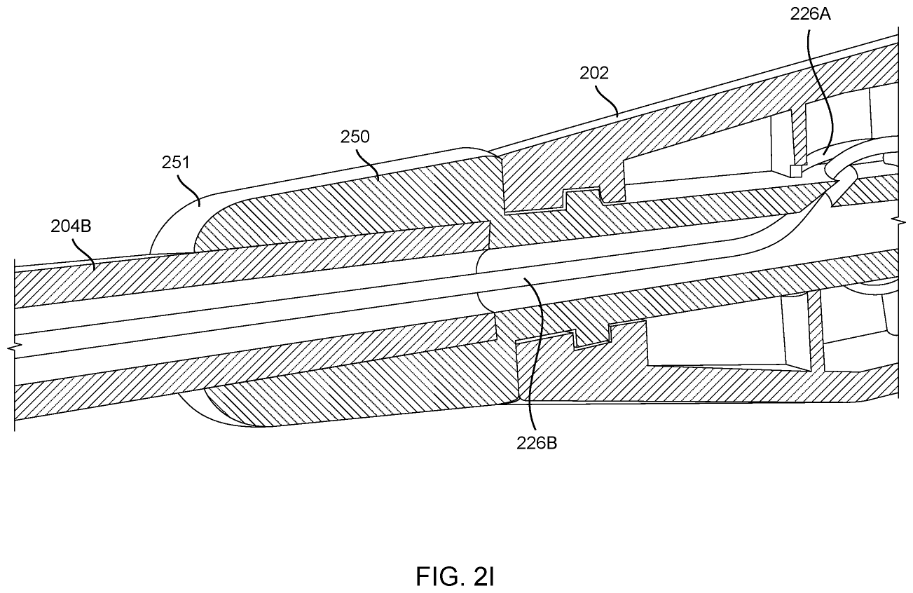

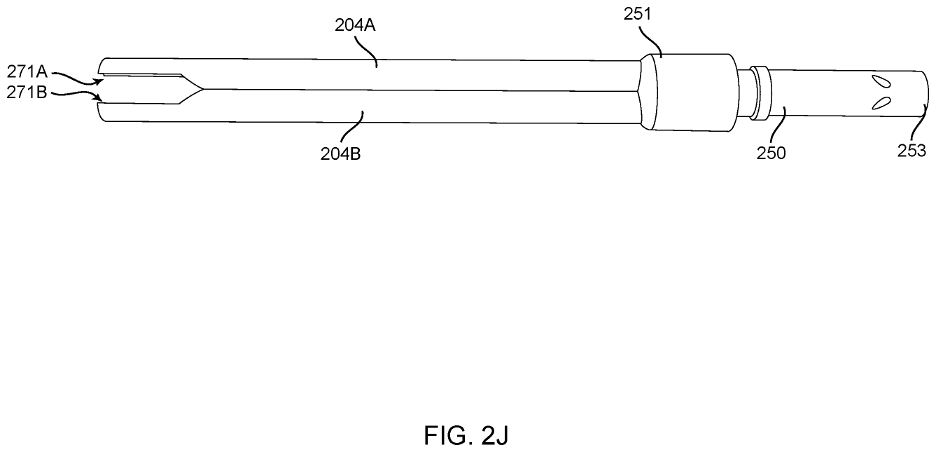

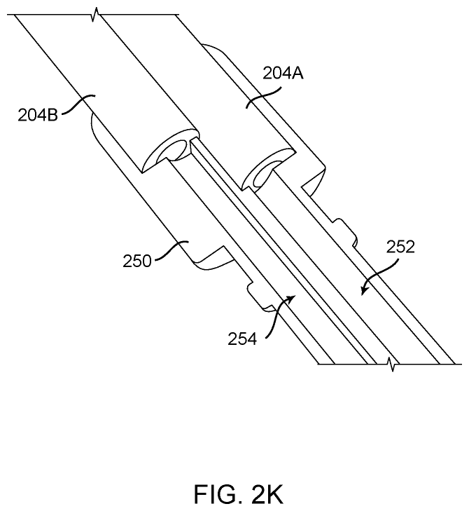

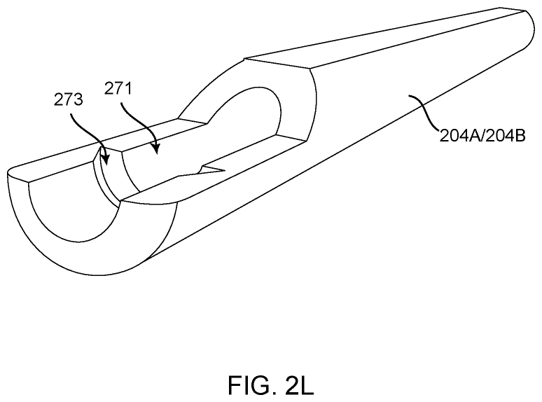

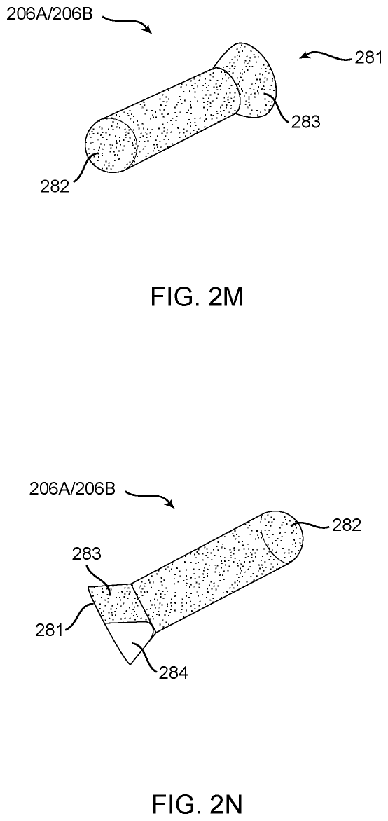

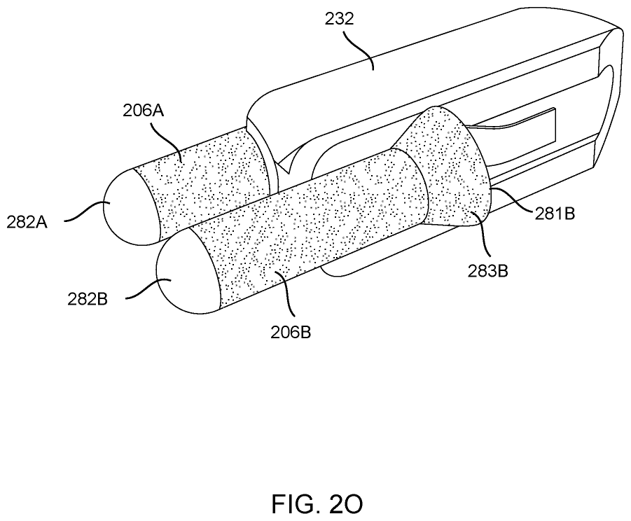

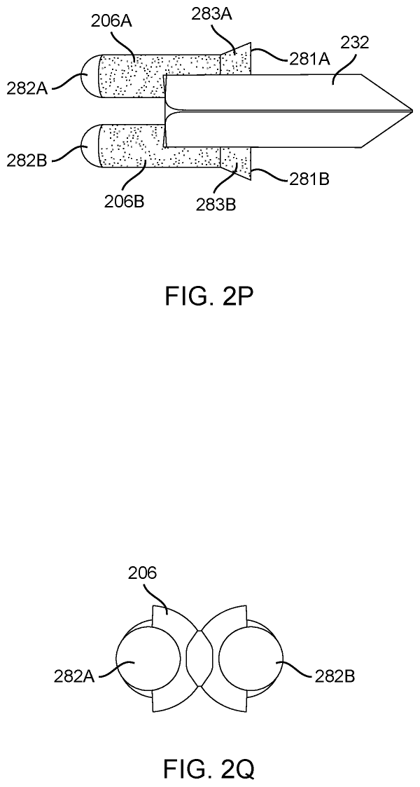

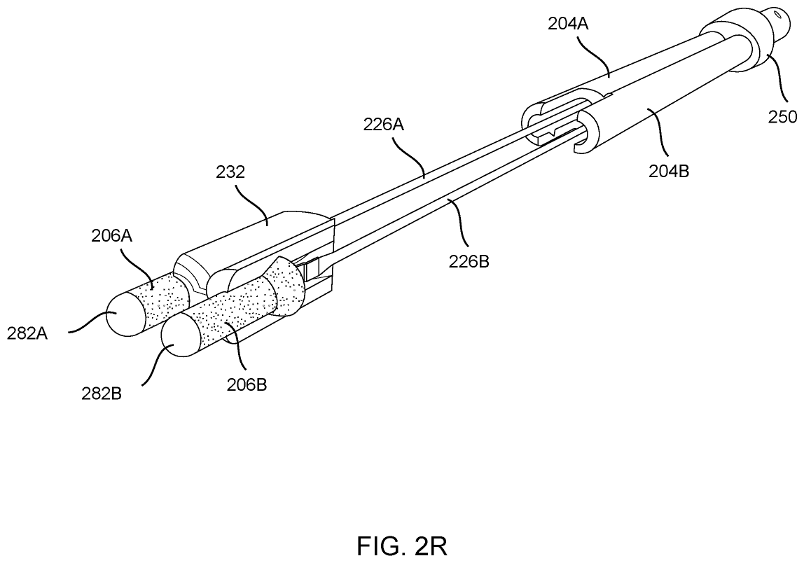

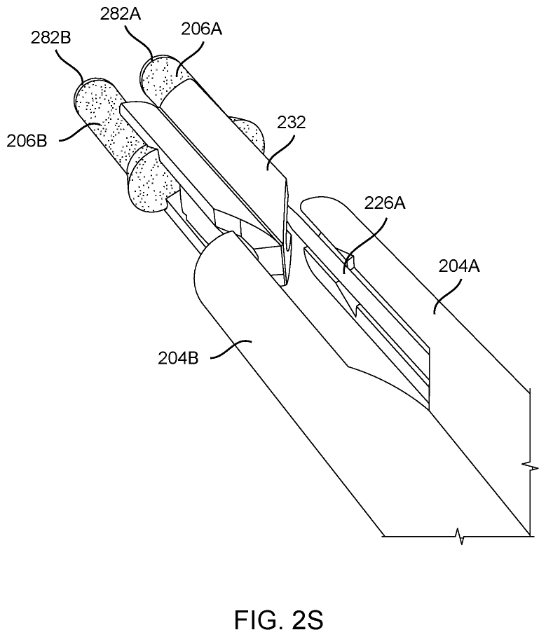

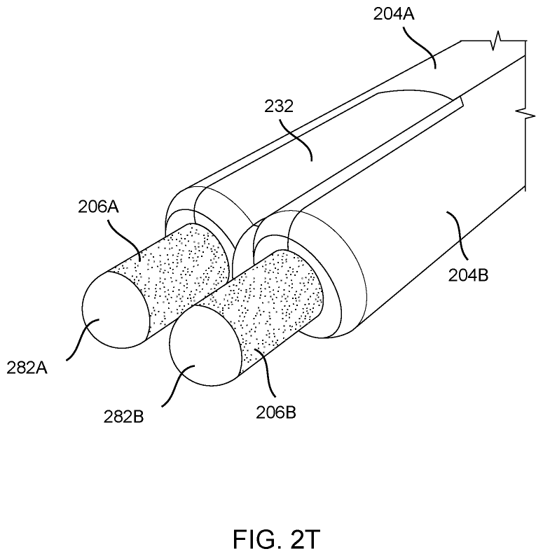

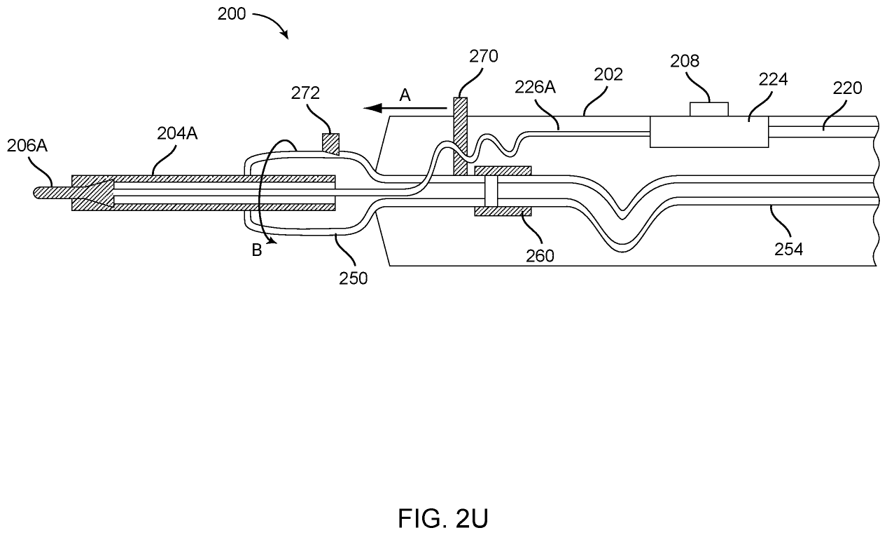

In another embodiment of the present disclosure, apparatus 100 may be modified for bipolar electrosurgical applications. For example, a bipolar electrosurgical apparatus 200 is shown in FIGS. 2A and 2B for use with the system 10 of the present disclosure. It is to be appreciated that, in the description of apparatus 200 that follows, elements of apparatus 200 that are similarly numbered to the elements of apparatus 100 are configured in a similar manner as described above unless specified otherwise.

Referring to FIGS. 2C and 2D, the components of apparatus 200 are shown in greater detail in accordance with the present disclosure. Apparatus 200 includes electrodes 206A, 206B, shaft 204, tip cap 232, coupling members 250, 260, circuit 224, button 208, slider 209, tube 254, and connector 230. Connector 230 is disposed through a proximal end of handle 202 and couples tube 54 to tube 254 for receiving a fluid (e.g., saline) via assembly 50. Tube 254 is further coupled to a y-connector tube 260, where connector 260 is further coupled connector 250, each which will be described in greater detail below. Connector 250 is coupled to tubes 204A, 204B of shaft 204. Cable 120 is disposed through a proximal end of handle 202 and couples an electrosurgical generator to circuit 224 of apparatus 200. Circuit 224 is further coupled to a wire 226A, where wire 226A is disposed through connector 250 and tube 204A and further coupled to electrode 206A. Wire 226B is through tube 204B and coupled to electrode 206B and cable 120.

Button 208 is disposed through a wall of handle 202 and configured to activate circuit 224 such that electrosurgical energy provided via cable 120 is applied to wire 226A and via wire 226 to electrode 206A. As described above, apparatus 200 is configured for bipolar applications. When each of electrodes 206A, 206B is in contact with tissue, energy is returned via electrode 206B and wire 226B to cable 120 to be provided to the electrosurgical generator.

It is to be appreciated that each of electrodes 206A, 206B are configured with material having a porous structure (e.g., in the manner described above with respect to electrode 106), such that when a fluid (e.g., saline) is provided through each of tubes 204A, 204B, the fluid passes through each of electrodes 206A, 206B and is applied to patient tissue.

Referring to FIGS. 2E and 2F, connector 250 is shown in accordance with the present disclosure. Connector 250 is configured to couple each of tubes 204A, 204B to handle 202. Connector 250 includes a proximal end 253 and a distal end 251. Separate fluid channels 252, 254 extend through the interior of connector 250 from end 253 to end 251. Connectors 250 further includes wires apertures 256, 258. Wire aperture 256 is configured to provide access to channel 254 and wire aperture 258 is configured to provide access to channel 252.

Referring to FIG. 2G, connector 260 is configured to be disposed around the proximal end 253 of connector 250 and the distal end of tube 254, such that fluid provided via tube 254 is further provided into each of channels 252, 254. Referring to FIGS. 2G, 2H and 2I, wire 226A is disposed through wire aperture 258 and channel 252 of connector 250 and out of distal end 251. Wire 226B is disposed through wire aperture 256 and channel 254 of connector 250 and out of distal end 251. The proximal end of tube 204A is disposed through end 251 of connector 250 and into channel 252, such that wire 226A extends into the interior of tube 204A. The proximal end of tube 204B is disposed through end 251 of connector 250 and into channel 254, such that wire 226B extends into the interior of tube 204B. Referring to FIGS. 2J and 2K, the distal ends of each of tubes 204A, 204B are shown disposed in channels 252, 254 respectively. As shown in FIGS. 2J, 2K, fluid provided via channel 252 enters the interior of tube 204A and is provided to electrode 206A and fluid provided via channel 254 enters the interior of tube 204B and is provided to electrode 206B.

Referring to FIGS. 2J and 2L, the distal ends of each of tubes 204A, 204B include a slot 271 configured to receive a respective one of electrodes 206A, 206B. Tubes 204A, 204B are coupled such that slot 271A of tubes 204A is oriented toward slot 271B of tubes 204B. Slot 271A is configured to receive a portion of electrode 206A and slot 271B is configured to receive a portion of electrode 206B. Referring to FIGS. 2M and 2N, electrodes 206A and 206B (which are each configured in the same manner) are shown. Each electrode 206 includes a proximal end 281 and a distal end 282. Proximal end 281 includes a substantially conic portion 283 having a planar surface 284. Distal end 282 is configured with a rounded or blunted end. In one embodiment, the distal end 282 of each electrode is configured in a round (hemispherical) shape and is solid (e.g., having a low and/or essentially zero porosity). The remaining portions of each electrode 206 are configured with a porous structure (e.g., the central cylindrical portion and the conic portion 283 of each electrode 206). In this way, fluid provided to each electrode 206 cannot escape via distal end 282 and can only escape via the remaining portions of each electrode 206 (e.g., the outer walls of the central cylindrical portion). Furthermore, as described above, because the distal end 282 of each electrode 206 is configured to be solid, a higher energy density develops at each end 282 when energy is applied across electrodes 206A, 206B. It is to be appreciated that, in other embodiments, electrodes 206 may be configured in other geometrical configurations (e.g., needles) without deviating from the scope of the present disclosure.

Referring again to FIGS. 2J and 2L, slots 271A, 271B of the distal ends of tubes 204A, 204B each includes a portion 273 dimensionsed to receive the conic portion 283 of each electrode 206. Referring to FIGS. 2O, 2P, 2Q, electrodes 206A, 206B are coupled to the distal ends of tubes 204A, 204B, respectively, via a molded (e.g., injection molded) cap 232. Molded cap 232 is dimensioned to fit between each of slots 271A, 271B. As shown in FIGS. 2R, 2S, 2T, molded cap 232 is coupled between the distal ends of tubs 204A, 204B and wires 226A, 226B are coupled to electrodes 206A, 206B, respectively. In one embodiment, the distal end of wire 225A is coupled to planar surface 284A of electrode 206A and the distal end wire 226B is coupled to planar surface 284B.

It is to be appreciated that, although in the embodiments above molded cap 232 is used to couple electrodes 206A, 206B to the distal ends of tubes 204A, 204B, electrodes 206A, 206B may be coupled to the distal ends of tubes 204A, 204B via other suitable methods. For example, in other embodiments of the present disclosure, electrodes 204A, 204B may be coupled to the distal ends of tubes 204A, 204B via heat staking, adhesive, or other suitable methods.

Referring to FIG. 2U, in one embodiment, apparatus 200 is configured such that shaft 204 is extendable distally or retractably proximally relative to handle 202. In this embodiment, connector 250 is coupled to handle 202, such that connector 250 is extendable or retractable relative to handle 202. A slider 270 is disposed through a slot in the outer wall of handle 202 and coupled to a proximal portion of the exterior of connector 250. By advancing slider 270 distally relative to handle 102 (as denoted by the letter A in FIG. 2U), shaft 204 and electrodes 206A, 206B are also advanced distally relative to handle 102. Alternatively, by retracting slider 270 proximally relative to handle 202 (e.g., in a direction opposite to direction A), shaft 204 and electrodes 206A, 206B are also retracted proximally relative to handle 202. It is to be appreciated that in this embodiment, wires 226A, 226B and fluid tube 254 are configured to be flexible and with a sufficient amount of slack to accommodate the distal extension and proximal retraction of shaft 204 and connector 250 relative to handle 202. In another embodiment tube 254 may be configured with a telescoping mechanism (e.g., one or more folds in a portion of the outer wall of tube 254) such that the length of tube 254 may be lengthened or shortened as necessary to accommodate the distal extension and proximal retraction of shaft 204 relative to handle 202. The embodiment of FIG. 2U advantageously enables a user to change the length of shaft 204, thus enabling apparatus 200 to be used for electrosurgical procedures requiring electrodes 206A, 206B to reach various depths within the cavity of the patient that shaft 204 is disposed through.

In some embodiments, connector 250 may be mounted to handle 202 such that connector 250 is rotatable relative to handle 202 (e.g., as denoted by the arrow labelled with the letter B in FIG. 2U) to change the orientation of electrodes 206A, 206B with respect to handle 202. In this embodiment, apparatus 200 further includes a knob or rotation actuation member 272 coupled to connector 250. Knob 272 is configured such that when knob 272 is rotated with respect to handle 202, connector 250, shaft 204 and electrodes 206A, 206B are also rotated with respect to handle 202, thus enabling a user to selectively change the orientation of electrodes 206A, 206B with respect to handle 202 without requiring a user to rotate handle 202.

In one embodiment, connector 250 and shafts 204A, 204B are rotatable relative to flow tube 254. In this embodiment, one or more seals may be provided to prevent fluid from leaking from apparatus 200 while shafts 204A, 204B and connector 250 are rotated. For example, a seal may be disposed between connector 250 and connector 260 to prevent leaking of fluid provided to shafts 204A, 204B while connector 250 and shafts 204A, 204B are rotated.

It is to be appreciated that in the embodiments described above, shaft 204 is configured to be rigid and linear. However, in other embodiments of the present disclosure, shaft 204 may be configured to be flexible to enable shaft 204 to be bent such that distal end of shaft 204 may achieve a variety of different orientations with respect to handle 202. In some embodiments, the distal end of shaft 204 may be configured to be grasped by forceps of a robotic arm to manipulate the orientation of the distal end of shaft 204 with respect to handle 202.

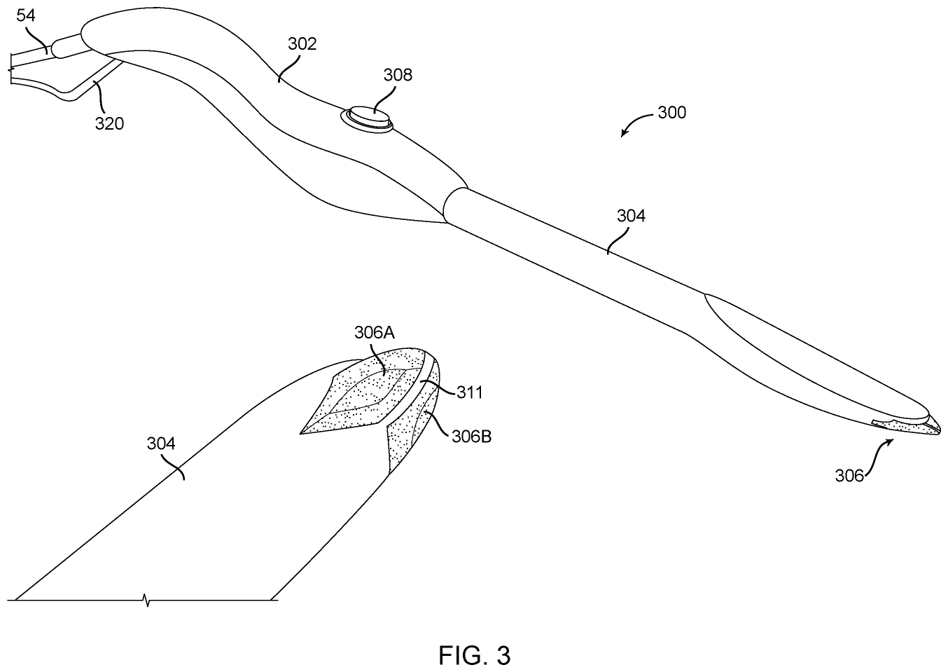

In another embodiment of the present disclosure, electrodes 206 may be bonded together using an insulative material. For example, referring to FIG. 3, a bipolar electrosurgical apparatus 300 is shown in accordance with the present disclosure. Apparatus 300 in includes a cable 320 (coupled to a connector, not shown), a handle or housing 302, a shaft 304, and a bipolar electrode 306 formed at the distal tip of apparatus 300. Handle 302 is coupled via cable 320 to an electrosurgical generator for receiving electrosurgical energy and to a fluid source via tube 54 for receiving a fluid (e.g., saline).

Electrode 306 is configured as a bipolar electrode having electrode 306A (e.g., an active electrode) and electrode 306B (e.g., a return electrode). In one embodiment, electrodes 306A, 306B are bonded together using an insulative material 311. Material 311 serves as both an electrically insulative spacer between electrodes 306A, 306B and a means for integrating electrodes 306A, 306B (and material 311) into a cohesive shape to form the distal tip/electrode 306. For example, in one embodiment, tip or electrode 306 is shaped as a tissue elevator configured for scraping tissue or raising tissue off of a bony surface. Electrodes 306A, 306B are each made of a material having a porous structure (as described above). In this way, when fluid is provided via tube 54 and shaft 304 to electrodes 306A, 306B the fluid is provided via the porous structure to patient tissue being treated. Although not shown, apparatus 300 includes a slider or other selection means for controlling the flow rate of the fluid. Furthermore, when button 308 is pressed electrosurgical energy received from the electrosurgical generator is applied to patient tissue across electrodes 306A, 306B.

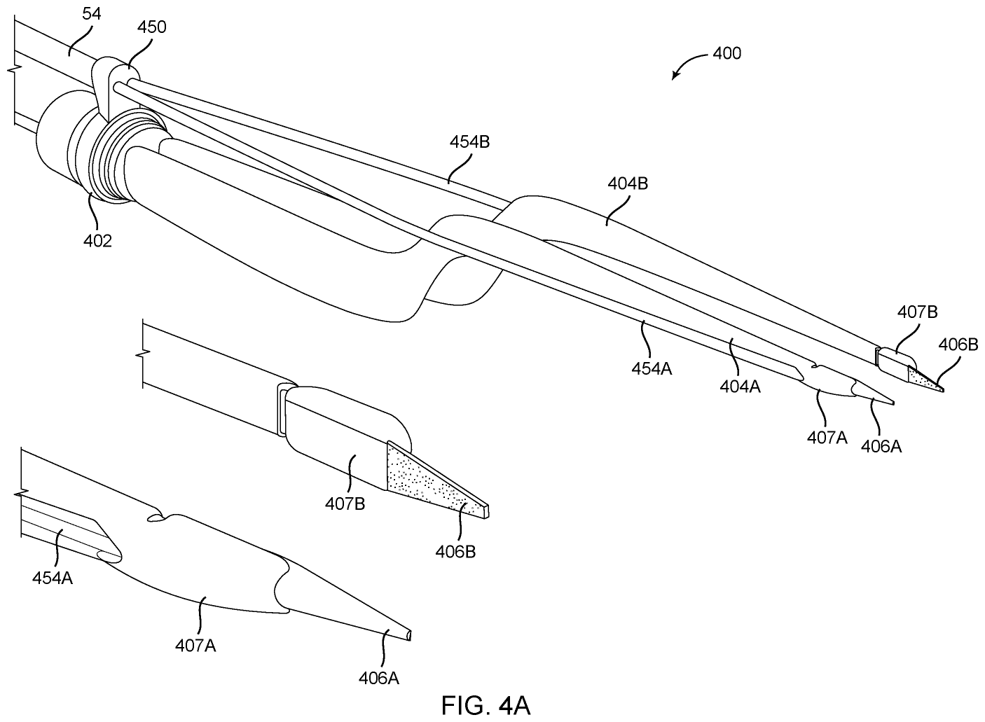

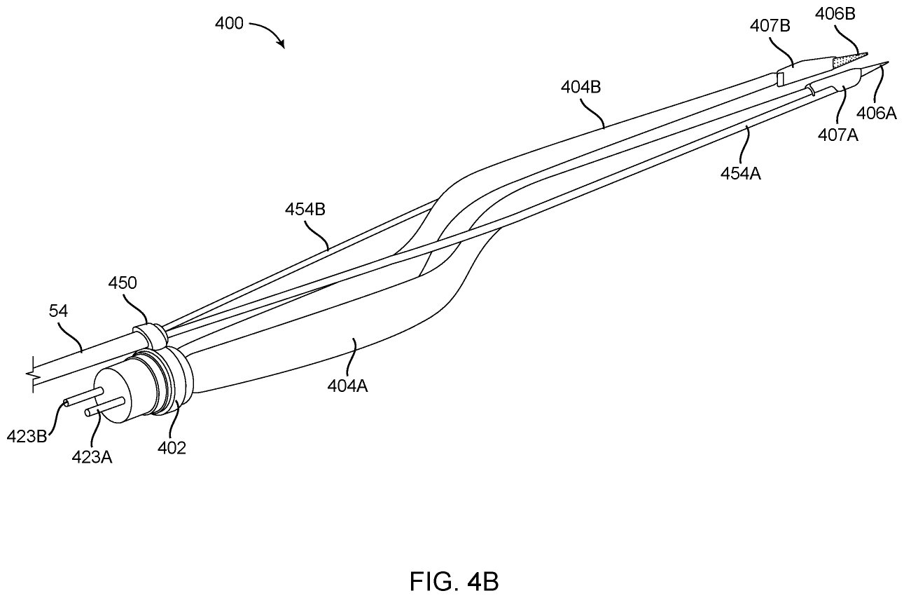

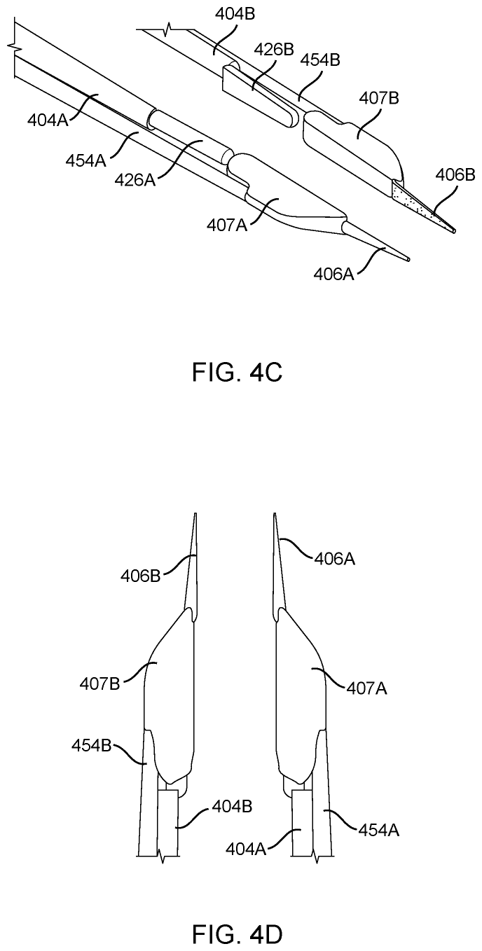

Referring to FIGS. 4A, 4B, in another embodiment, an electrosurgical apparatus 400 is provided that is configured as bipolar electrosurgical forceps. Apparatus 400 includes a base 402, elongated prongs or legs 402A, 402B, fluid tubes 454A, 454B, tip housings 407A, 407B, and electrodes 406A, 406B. Electrodes 406A, 406B are each made of a material having a porous structure, as described above.

The proximal ends of each of prongs 404A, 404B are coupled to base 402. The distal ends of each of prongs 404A, 404B are coupled to respective tip housings 407A, 407B. It is to be appreciated that, in one embodiment, tip housings 407A, 407B are integrated into the distal ends of each of prongs 404A, 404B. Electrode 406A is coupled to the distal end of prong 404A and electrode 406B is coupled to the distal end of prong 404B. In one embodiment, each of electrodes 406A, 406B includes a pointed distal end and a flat or planar surface extending along a side of each electrode 406A, 406B. Electrodes 406A, 406B are coupled to prongs 404A, 404B, respectively, such that the flat or planar surface of electrode 406A faces or is oriented toward the flat or planar surface of electrode 406B to facilitate the gripping of tissue between each of the planar surfaces.

Base 402 includes terminals or electric leads 423A, 423B, which are configured to be coupled to an electrosurgical generator (or a cable/conductor coupled to an electrosurgical generator) for receiving electrosurgical energy. Terminal 423A is coupled to a conductor 426A, which extends internally from the proximal end of prong 404A to the distal end of 404A and into tip housing 407A, where conductor 426A is coupled to electrode 406A. Terminal 423B is coupled to a conductor 426B, which extends internally from the proximal end of prong 404B to the distal end of 404B and into tip housing 407B, where conductor 426B is coupled to electrode 406B. In this way, electrosurgical energy received from an electrosurgical generator is provided to one of electrodes 406A, 406B, via terminals 423A, 423B and conductors 426A, 426B. The other of electrodes 406A, 406B is configured as a return electrode. In one embodiment, apparatus 400 may include a button to control the application of electrosurgical energy to electrodes 406A, 406B.

Base 402 includes a connector 450 configured to be coupled to fluid tube 54 for receiving a fluid (e.g., saline) from a fluid source. Connector 450 is further coupled to tubes 454A, 454B, where tube 454A is further coupled to electrode 406A via tip housing 407A and tube 454B is further coupled to electrode 407B via tip housing 407B. Connector 450 is configured to split the fluid received via tube 54 into separate fluid streams, where a first fluid stream is provided through tube 454A to tip housing 407A and electrode 406A and a second fluid stream is provided through tube 454B to tip housing 407B and electrode 407B. Since, as described above, each of electrodes 406A, 406B is made of a conductive material having a porous structure, fluid provided to each of tip housings 407A, 407B is provided through the porous structures of electrodes 406A, 406B to patient tissue adjacent to each of electrodes 406A, 406B.

In one embodiment, as shown in FIGS. 4A-4C, the flat planar surfaces of electrodes 406A, 406B that face each other are configured to have a predetermined porosity, while the remaining surfaces of electrodes 406A, 406B (i.e., the portions that do not face each other) are configured to be solid, with zero porosity. In this way, in these embodiments, fluid may only escape via the flat surfaces of electrodes 406A, 406B, which face each other. In other embodiments, the entire surface of each of electrodes 406A, 406B is configured to be porous to enable fluid to escape all around electrodes 406A, 406B.

Prongs 404A, 404B are configured to enable a user to move prongs 404A, 404B relative to each other to advance electrodes 406A, 406B toward or away from each other. Prongs 404A, 404B are coupled via base 450 such that prongs 404A, 404B are biased away from each other and the distal ends of prongs 404A, 404B and electrodes 406A, 406B are spaced or separated by a predetermined distance. When the distal ends of prongs 404A, 404B separated, forceps 400 are in an open position. A user may bring electrodes 406A, 406B toward each other to grasp patient tissue during a procedure by applying pressure on an outer surface of each of prongs 404A, 404B (e.g., while gripping the proximal ends of prongs 404A, 404B) to cause forceps 400 to achieve a closed position. While patient tissue is grasped between electrodes 406A, 406B, electrosurgical energy received from an electrosurgical generator (or other energy source coupled to terminals 423A, 423B) is applied across electrodes 406A, 406B and a fluid received from a fluid source is provided via electrodes 406A, 406B to the patient tissue being treated to cause a desired electrosurgical effect (e.g., ablation, etc.)

It is to be appreciated that, in any of the electrosurgical apparatuses described above, one or more of the included components (e.g., electrodes, shafts, and/or components therebetween) may be coupled together via one or more techniques, including, but not limited to, using one or more adhesive agents, ultrasonic welding, threaded connections, injection molding, and/or any other suitable techniques.

In one aspect of the present disclosure, an electrosurgical apparatus is provided comprising: a handle; a shaft coupled to and extending from the handle, the shaft including a distal end; and at least one porous electrode coupled to the distal end of the shaft, the at least one porous electrode including a porous structure configured to enable a fluid provided to the distal end of the shaft to flow through the porous structure and exit the at least one porous electrode.

In one aspect, the electrosurgical apparatus is provided, wherein the at least one porous electrode is made of a conductive material configured to conduct electrosurgical energy provided to the distal end of the shaft.

In one aspect, the electrosurgical apparatus is provided, wherein the electrosurgical apparatus is configured as a monopolar device and the at least one porous electrode is configured as an active electrode.

In one aspect, the electrosurgical apparatus is provided, wherein the at least one porous electrode includes a first porous electrode and a second porous electrode and the electrosurgical apparatus is configured as a bipolar device.

In one aspect, the electrosurgical apparatus is provided, wherein the shaft includes a first channel and a second channel, the first porous electrode coupled to the first channel for receiving the fluid and the second porous electrode coupled to the second channel for receiving the fluid.

In one aspect, the electrosurgical apparatus is provided, wherein each porous electrode includes a blunted distal end and a central cylindrical portion, the blunted distal end of each porous electrode configured to be solid having zero porosity and the central cylindrical portion of each electrode includes the porous structure such that the fluid provided to each electrode only exits from the central cylindrical portion including the porous structure.

In one aspect, the electrosurgical apparatus is provided, wherein the first porous electrode and the second porous electrode are bonded together using an insulative material to form a distal tip having a cohesive shape.

In one aspect, the electrosurgical apparatus is provided, wherein the distal tip is shaped as a tissue elevator configured for scraping or raising tissue off of a surface.

In one aspect, the electrosurgical apparatus is provided, wherein the shaft is rotatable relative to the handle to enable the orientation of the electrode with respect to the handle to be changed.

In one aspect, the electrosurgical apparatus is provided, wherein the shaft is extendable and retractable relative to the handle.

In one aspect, the electrosurgical apparatus is provided, wherein the shaft is flexible.

In one aspect, the electrosurgical apparatus is provided, wherein the at least one porous electrode includes a concave edge.

In one aspect, the electrosurgical apparatus is provided, wherein the at least one porous electrode includes a first convex surface and a second opposite convex surface, the first convex surface and second convex surface sharing a sharpened edge.

In one aspect, the electrosurgical apparatus is provided, wherein the at least one porous electrode is configured as a blade including at least one beveled surface forming a sharp edge.

In one aspect, the electrosurgical apparatus is provided, wherein the at least one beveled surface is configured to be solid having zero porosity and portions of the at least one porous electrode other than the at least one beveled surface are configured from the porous structure.

In one aspect, the electrosurgical apparatus is provided, wherein the at least one porous electrode includes at least one protrusion and a distal portion of the shaft includes at least one slot configured to receive the protrusion, such that when the electrode is coupled to distal end of the shaft and the at least one protrusion is disposed in the slot, the electrode is prevented from being separated from the shaft.

In one aspect, the electrosurgical apparatus is provided, further comprising at least one flow controller disposed on the handle and configured to control a flow rate of the fluid provided to the distal end of the shaft.

In one aspect, the electrosurgical apparatus is provided, wherein the fluid is saline.

In one aspect, the electrosurgical apparatus is provided, wherein the porous structure is microporous.

In one aspect, the electrosurgical apparatus is provided, wherein the porous structure is macroporous.

In one aspect, the electrosurgical apparatus is provided, wherein the porous structure is hydrophilic.

In one aspect, the electrosurgical apparatus is provided, wherein two or more regions of the at least one porous electrode are configured with different levels of porosity relative to each other.

In one aspect, the electrosurgical apparatus is provided, wherein at least one region of the at least one porous structure is configured with zero porosity, such that no fluid may exit through the at least one region.

In one aspect, the electrosurgical apparatus is provided, wherein the porous structure is made of a metallic material.

In one aspect, the electrosurgical apparatus is provided, wherein the porous structure is made of a non-metallic material.

In one aspect, the electrosurgical apparatus is provided, wherein at least one first region of the porous structure is made of a conductive material and at least one second region of the porous structure is made of a non-conductive material.

In one aspect, the electrosurgical apparatus is provided, wherein the at least one porous electrode is coupled to the shaft via a threaded connection.

In one aspect, the electrosurgical apparatus is provided, further comprising a threaded connection member is configured to couple the at least one porous electrode to the shaft.

In one aspect, the electrosurgical apparatus is provided, wherein the threaded connection member includes male threads and the at least one porous electrode includes female threads, the female threads of the at least one porous electrode configured to mate with the male threads of the threaded connection member to couple the at least one porous electrode to the threaded connection member.

In one aspect, the electrosurgical apparatus is provided, wherein the threaded connection member includes female threads and the distal end of the shaft includes male threads, the female threads of the threaded connection member configured to mate with the male threads of the distal end of the shaft to couple the threaded connection member to the distal end of the shaft.

In one aspect, the electrosurgical apparatus is provided, wherein the threaded connection member includes a channel configured to provide fluid received from the shaft to the at least one porous electrode.

In one aspect, the electrosurgical apparatus is provided, wherein the shaft is made of a conductive material and the shaft is configured to provided electrosurgical energy to the at least one porous electrode.

In one aspect, the electrosurgical apparatus is provided, wherein the threaded connection member is made of a conducting material.

In one aspect, the electrosurgical apparatus is provided, wherein the threaded connection member is made of a non-conductive material and the at least one porous electrode is coupled to the shaft via at least one conductor.

In one aspect, the electrosurgical apparatus is provided, wherein the shaft includes a foam material configured to enable fluid to flow through the foam material to be provided to the at least one porous electrode.

In one aspect, the electrosurgical apparatus is provided, further comprising a conductor disposed through the foam material within the shaft and coupled to the at least one porous electrode for providing electrosurgical energy thereto.

In one aspect, the electrosurgical apparatus is provided, wherein the conductor includes a distal end having threads and the at least one porous electrode includes a proximal end having threads, the threads of the conductor mating with the threads of the at least one porous electrode to couple the conductor to the at least one porous electrode.

In one aspect, the electrosurgical apparatus is provided, wherein the at least one porous electrode is coupled to the distal end of the shaft by injection molding a cap over at least a portion of the at least one porous electrode and the distal end of the shaft.

In one aspect, the electrosurgical apparatus is provided, wherein the handle is configured to be coupled to a fluid assembly for receiving the fluid.

In another aspect of the present disclosure, bipolar electrosurgical forceps are provided comprising: first and second prongs, each prong including a proximal end and a distal end; a first porous electrode coupled to the distal end of the first prong; a second porous electrode coupled to the distal end of the second prong; each porous electrode including a porous structure configured to enable a fluid provided to the distal end of each prong to flow through the porous structure and exit the respective electrode; wherein the first prong and the second prong are configured to move relative to each other to enable the first porous electrode and second porous electrode to be advanced toward each other to grip patient tissue such that electrosurgical energy received from an energy source is applied across the first porous electrode and the second porous electrode to the patient tissue.

In one aspect, the bipolar electrosurgical forces are provided, wherein the first porous electrode is configured as an active electrode.

In one aspect, the bipolar electrosurgical forces are provided, wherein the second porous electrode is configured as a return electrode.

In one aspect, the bipolar electrosurgical forces are provided, further comprising a base including a first terminal and a second terminal, the first and second terminal configured to be coupled to the energy source to receive electrosurgical energy, wherein each prong includes a conductor and the proximal ends of the first and second prong are each coupled to the base such that the conductor of the first prong is coupled to the first porous electrode and the first terminal and the conductor of the second prong is coupled to the second porous electrode and the second terminal, the first terminal and the second terminal are configured to be coupled to an energy source for receiving electrosurgical energy.

In one aspect, the bipolar electrosurgical forces are provided, further comprising a first tube and a second tube each coupled to the base and configured to receive a fluid, the first tube further coupled to the first porous electrode and configured to provide the fluid to the first porous electrode, the second tube further coupled to the second porous electrode and configured to provide the fluid to the second porous electrode.

In one aspect, the bipolar electrosurgical forces are provided, wherein the base includes a connector that is coupled to the first tube and the second tube, the connector configured to split the received fluid into first and second fluid streams, the first fluid stream provided through the first tube to the first porous electrode and the second fluid stream provided through the second tube to the second porous electrode.

In one aspect, the bipolar electrosurgical forces are provided, wherein the connector is configured to be coupled to a third tube, the third tube providing the fluid to the base from a fluid source.

In one aspect, the bipolar electrosurgical forces are provided, further comprising a first tube and a second tube, the first tube coupled to the first porous electrode and configured to provide a fluid to the first porous electrode, the second tube coupled to the second porous electrode and configured to provide the fluid to the second porous electrode.

In one aspect, the bipolar electrosurgical forces are provided, wherein the fluid is saline.

In one aspect, the bipolar electrosurgical forces are provided, wherein the porous structure of the first porous electrode and the second porous electrode is microporous.

In one aspect, the bipolar electrosurgical forces are provided, wherein the porous structure of the first porous electrode and the second porous electrode is macroporous.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. An electrosurgical apparatus comprising:
a handle;
a shaft coupled to and extending from the handle, the shaft including a distal end; and
at least one porous electrode coupled to the distal end of the shaft, the at least one porous electrode including a porous structure configured to enable a fluid provided to the distal end of the shaft to flow through the porous structure and exit the at least one porous electrode.

2. The electrosurgical apparatus of claim 1, wherein the at least one porous electrode is made of a conductive material configured to conduct electrosurgical energy provided to the distal end of the shaft.

3. The electrosurgical apparatus of claim 2, wherein the electrosurgical apparatus is configured as a monopolar device and the at least one porous electrode is configured as an active electrode.

4. The electrosurgical apparatus of claim 2, wherein the at least one porous electrode includes a first porous electrode and a second porous electrode and the electrosurgical apparatus is configured as a bipolar device.

5. The electrosurgical apparatus of claim 4, wherein the shaft includes a first channel and a second channel, the first porous electrode coupled to the first channel for receiving the fluid and the second porous electrode coupled to the second channel for receiving the fluid.

6. The electrosurgical apparatus of claim 5, wherein each porous electrode includes a blunted distal end and a central cylindrical portion, the blunted distal end of each porous electrode configured to be solid having zero porosity and the central cylindrical portion of each electrode includes the porous structure such that the fluid provided to each electrode only exits from the central cylindrical portion including the porous structure.

7. The electrosurgical apparatus of claim 4, wherein the first porous electrode and the second porous electrode are bonded together using an insulative material to form a distal tip having a cohesive shape.

8. The electrosurgical apparatus of claim 7, wherein the distal tip is shaped as a tissue elevator configured for scraping or raising tissue off of a surface.

9. The electrosurgical apparatus of claim 1, wherein the shaft is rotatable relative to the handle to enable the orientation of the electrode with respect to the handle to be changed.

10. The electrosurgical apparatus of claim 1, wherein the shaft is extendable and retractable relative to the handle.

11. The electrosurgical apparatus of claim 1, wherein the shaft is flexible.

12. The electrosurgical apparatus of claim 1, wherein the at least one porous electrode includes a concave edge.

13. The electrosurgical apparatus of claim 1, wherein the at least one porous electrode includes a first convex surface and a second opposite convex surface, the first convex surface and second convex surface sharing a sharpened edge.

14. The electrosurgical apparatus of claim 1, wherein the at least one porous electrode is configured as a blade including at least one beveled surface forming a sharp edge.

15. The electrosurgical apparatus of claim 14, wherein the at least one beveled surface is configured to be solid having zero porosity and portions of the at least one porous electrode other than the at least one beveled surface are configured from the porous structure.

16. The electrosurgical apparatus of claim 1, wherein the at least one porous electrode includes at least one protrusion and a distal portion of the shaft includes at least one slot configured to receive the protrusion, such that when the electrode is coupled to distal end of the shaft and the at least one protrusion is disposed in the slot, the electrode is prevented from being separated from the shaft.

17. The electrosurgical apparatus of claim 1, further comprising at least one flow controller disposed on the handle and configured to control a flow rate of the fluid provided to the distal end of the shaft.

18. The electrosurgical apparatus of claim 1, wherein the fluid is saline.

19. The electrosurgical apparatus of claim 1, wherein the porous structure is microporous.

20. The electrosurgical apparatus of claim 1, wherein the porous structure is macroporous.

21. The electrosurgical apparatus of claim 1, wherein the porous structure is hydrophilic.

22. The electrosurgical apparatus of claim 1, wherein two or more regions of the at least one porous electrode are configured with different levels of porosity relative to each other.

23. The electrosurgical apparatus of claim 22, wherein at least one region of the at least one porous structure is configured with zero porosity, such that no fluid may exit through the at least one region.

24. The electrosurgical apparatus of claim 1, wherein the porous structure is made of a metallic material.

25. The electrosurgical apparatus of claim 1, wherein the porous structure is made of a non-metallic material.

26. The electrosurgical apparatus of claim 1, wherein at least one first region of the porous structure is made of a conductive material and at least one second region of the porous structure is made of a non-conductive material.

27. The electrosurgical apparatus of claim 1, wherein the at least one porous electrode is coupled to the shaft via a threaded connection.

28. The electrosurgical apparatus of claim 27, further comprising a threaded connection member is configured to couple the at least one porous electrode to the shaft.

29. The electrosurgical apparatus of claim 28, wherein the threaded connection member includes male threads and the at least one porous electrode includes female threads, the female threads of the at least one porous electrode configured to mate with the male threads of the threaded connection member to couple the at least one porous electrode to the threaded connection member.

30. The electrosurgical apparatus of claim 28, wherein the threaded connection member includes female threads and the distal end of the shaft includes male threads, the female threads of the threaded connection member configured to mate with the male threads of the distal end of the shaft to couple the threaded connection member to the distal end of the shaft.

31. The electrosurgical apparatus of claim 28, wherein the threaded connection member includes a channel configured to provide fluid received from the shaft to the at least one porous electrode.

32. The electrosurgical apparatus of claim 28, wherein the shaft is made of a conductive material and the shaft is configured to provided electrosurgical energy to the at least one porous electrode.

33. The electrosurgical apparatus of claim 32, wherein the threaded connection member is made of a conducting material.

34. The electrosurgical apparatus of claim 32, wherein the threaded connection member is made of a non-conductive material and the at least one porous electrode is coupled to the shaft via at least one conductor.

35. The electrosurgical apparatus of claim 1, wherein the shaft includes a foam material configured to enable fluid to flow through the foam material to be provided to the at least one porous electrode.

36. The electrosurgical apparatus of claim 35, further comprising a conductor disposed through the foam material within the shaft and coupled to the at least one porous electrode for providing electrosurgical energy thereto.

37. The electrosurgical apparatus of claim 36, wherein the conductor includes a distal end having threads and the at least one porous electrode includes a proximal end having threads, the threads of the conductor mating with the threads of the at least one porous electrode to couple the conductor to the at least one porous electrode.

38. The electrosurgical apparatus of claim 1, wherein the at least one porous electrode is coupled to the distal end of the shaft by injection molding a cap over at least a portion of the at least one porous electrode and the distal end of the shaft.

39. The electrosurgical apparatus of claim 1, wherein the handle is configured to be coupled to a fluid assembly for receiving the fluid.

40. Bipolar electrosurgical forceps comprising:
first and second prongs, each prong including a proximal end and a distal end;
a first porous electrode coupled to the distal end of the first prong;
a second porous electrode coupled to the distal end of the second prong;
each porous electrode including a porous structure configured to enable a fluid provided to the distal end of each prong to flow through the porous structure and exit the respective electrode;
wherein the first prong and the second prong are configured to move relative to each other to enable the first porous electrode and second porous electrode to be advanced toward each other to grip patient tissue such that electrosurgical energy received from an energy source is applied across the first porous electrode and the second porous electrode to the patient tissue.

41. The bipolar electrosurgical forceps of claim 40, wherein the first porous electrode is configured as an active electrode.

42. The bipolar electrosurgical forceps of claim 41, wherein the second porous electrode is configured as a return electrode.

43. The bipolar electrosurgical forceps of claim 40, further comprising a base including a first terminal and a second terminal, the first and second terminal configured to be coupled to the energy source to receive electrosurgical energy, wherein each prong includes a conductor and the proximal ends of the first and second prong are each coupled to the base such that the conductor of the first prong is coupled to the first porous electrode and the first terminal and the conductor of the second prong is coupled to the second porous electrode and the second terminal, the first terminal and the second terminal are configured to be coupled to an energy source for receiving electrosurgical energy.

44. The bipolar electrosurgical forceps of claim 43, further comprising a first tube and a second tube each coupled to the base and configured to receive a fluid, the first tube further coupled to the first porous electrode and configured to provide the fluid to the first porous electrode, the second tube further coupled to the second porous electrode and configured to provide the fluid to the second porous electrode.

45. The bipolar electrosurgical forceps of claim 44, wherein the base includes a connector that is coupled to the first tube and the second tube, the connector configured to split the received fluid into first and second fluid streams, the first fluid stream provided through the first tube to the first porous electrode and the second fluid stream provided through the second tube to the second porous electrode.

46. The bipolar electrosurgical forceps of claim 45, wherein the connector is configured to be coupled to a third tube, the third tube providing the fluid to the base from a fluid source.

47. The bipolar electrosurgical forceps of claim 40, further comprising a first tube and a second tube, the first tube coupled to the first porous electrode and configured to provide a fluid to the first porous electrode, the second tube coupled to the second porous electrode and configured to provide the fluid to the second porous electrode.

48. The bipolar electrosurgical forceps of claim 40, wherein the fluid is saline.

49. The bipolar electrosurgical forceps of claim 40, wherein the porous structure of the first porous electrode and the second porous electrode is microporous.

50. The bipolar electrosurgical forceps of claim 40, wherein the porous structure of the first porous electrode and the second porous electrode is macroporous.
